SEQUENCE LISTING (8 sequences)

SEQ ID NO: 1 - protein, 15 aa, Homo sapiens
  MISC_FEATURE (13)..(15): Where GAA is GLU
SERTYRTRPILEGAA

SEQ ID NO: 2 - protein, 51 aa, Homo sapiens
  MISC_FEATURE (1)..(3): where GAA is GLU
GAAILETTACCGGLYSERLYSLYSTHRASNTYRASNGAALYSPHELYSGLY

SEQ ID NO: 3 - protein, 36 aa, Homo sapiens
TYRTYRTYRARGASNASPASPTYRGLYMETASPTYR

SEQ ID NO: 4 - protein, 33 aa, Homo sapiens
  MISC_FEATURE (28)..(30): Where TTA is LEU
SERALASERGLNASPILEARGASNTYRTTAASN

SEQ ID NO: 5 - protein, 21 aa, Homo sapiens
  MISC_FEATURE (13)..(15): Where TTA is LEU
TYRTHRSERTHRTTAHISSER

SEQ ID NO: 6 - protein, 27 aa, Homo sapiens
  MISC_FEATURE (19)..(21): where CCG is PRO
GLNGLNTYRSERLYSILECCGTYRTHR

SEQ ID NO: 7 - protein, 363 aa, Homo sapiens
  MISC_FEATURE (10)..(12): where TTA is LEU
GLNVALGLNTTAGLNGLNSERGLYALAGAGTTAMETLYSCCGGLYALASERVALLYSILESERCYSLYSALATHRGLYTYRTHRPHESERSERTYRTRPILEGAGTRPVALLYSGLNARGCCGGLYHISGLYTTAGAGTRPILEGLYGAGILETTACCGGLYSERLYSLYSTHRASNTYRASNGAGLYSPHELYSGLYLYSALATHRPHETHRALAASPTHRSERSERASNTHRALATYRMETGLNPHESERSERTTATHRSERGAGASPSERALAVALTYRTYRCYSALAARGTYRTYRTYRARGASNASPASPTYRGLYMETASPTYRTRPGLYGLNGLYTHRSERVALTHRVALSERSER

SEQ ID NO: 8 - protein, 336 aa, Homo sapiens
  MISC_FEATURE (1)..(3): Where GAG is GLU
GAGILEHISMETTHRGLNTHRTHRSERSERTTASERALASERTTAGLYASPARGVALTHRILESERCYSSERALASERGLNASPILEARGASNTYRTTAASNTRPTYRGLNGLNLYSCCGASPGLYTHRVALLYSTTATTAILETYRTYRTHRSERTHRTTAHISSERGLYVALCCGSERARGPHESERGLYSERGLYSERGLYTHRASPTYRSERTTATHRILESERASNTTAGAGCCGGAGASPILEALATHRTYRTYRCYSGLNGLNTYRSERLYSILECCGTYRTHRPHETHRGLYGLYTHRLYSTTAGAGILELYSARGALAASPALAALA

(19) United States
(12) Patent Application Publication
(10) Pub. No.: US 2016/0008466 A1
(43) Pub. Date: Jan. 14, 2016

(54) PARTICLE COMPOSITIONS AND METHODS RELATED THERETO

(71) Applicant: EMORY UNIVERSITY, Atlanta, GA (US)

(72) Inventor: Constantinos G. HADJIPANAYIS, Decatur, GA (US)

(73) Assignee: EMORY UNIVERSITY, Atlanta, GA (US)

(21) Appl. No.: 14/768,335

(22) PCT Filed: Feb. 24, 2014
(86) PCT No.: PCT/US2014/017976

§ 371 Date: Aug. 17, 2015

Related U.S. Application Data
(60) Provisional application No. 61/769,226, filed on Feb. 26, 2013.

(51) Int. Cl. A61K 41/00; A61N 2/00; C07K 16/30; A61K 47/48; A61K 45/06

(52) U.S. Cl. CPC A61K 41/0052; A61K 47/48861; A61K 47/48884; A61K 45/06; C07K 16/3053; A61N 2/002; C07K 2317/24

(57) ABSTRACT

The disclosure relates to metal particles compositions and uses related thereto. In certain embodiments, the disclosure relates to metal particles such as iron particles and their application in imaging and treating cancer.

### CROSS REFERENCE TO RELATED APPLICATIONS

This application is a 371 U.S.C. of International Patent Application No. PCT/US2014017976 filed Feb. 24, 2014, which claims the benefit of priority to U.S. Provisional Application No. 61/769,226 filed Feb. 26, 2013, which applications are hereby incorporated by reference in their entireties.

### STATEMENT REGARDING FEDERALLY FUNDED RESEARCH

This invention was made with government support under Grant Nos. NS053454 and P50CA128301-01A10003 awarded by the National Institutes of Health. The government has certain rights in the invention.

### FIELD

The disclosure relates to metal particles compositions and uses related thereto. In certain embodiments, the disclosure relates to iron particles and their application in imaging and treating cancer.

### BACKGROUND

Despite the use of conventional therapeutic modalities such as surgery, chemotherapy, and ionizing radiation, the prognosis in patients with malignant gliomas remains poor. Most glioblastoma multiforme (GBM) tumors recur at the site of their initial treatment due to the presence of infiltrating cancer cells in the surrounding normal brain that resist therapy or go untreated. Infiltrating cancer cells include a subpopulation of glioblastoma stem cells (GSC) shown to be integral to tumor development, perpetuation, and therapy resistance. Imaging and targeted therapy of infiltrating GBM cells within the normal brain remain limited.

Accurate targeting and better delivery efficiency are two major goals in the development of therapeutic agents for the treatment of malignant brain tumors. Ideally, a therapeutic agent would be able to overcome the BBB and selectively be enriched in tumors with minimal toxicity to normal tissues. Conjugation of agents to antibodies or other ligands that bind to antigens or receptors that are usually abundantly or uniquely expressed on the tumor surface represents an approach in the treatment of glioblastoma tumors. Kunwar et al., J Clin Oncol, 2007, 25:837-44 and Sampson et al., Neuro Oncol, 2008, 10:320-9.

Convection-enhanced delivery CED is an approach developed to overcome the obstacles associated with current CNS agent delivery and is increasingly used to distribute therapeutic agents for treatment of malignant gliomas. Hadjipanayis et al., Mol Ther, 2008, 16:1783-8. Clinical trials have been performed using CED for the treatment of recurrent GBM. In CED, a small hydrostatic pressure differential is imposed by a syringe pump to distribute infusate directly to small or large regions of the CNS. Difficulty in CED imaging of therapeutic agents in the brain was limitation of this approach. Groups have radiolabeled their therapeutic agent, co-infused their agent with radiolabeled albumin (123I-labeled albumin), or used liposomes containing an MRI contrast agent (e.g., gadoteridol) for CED imaging. Sampson et al. Neuro Oncol 2008; 10:320-9. Radiolabeling of therapeutic agents relies on single-photon emission computerized tomography for agent imaging, which is a low-resolution imaging modality of the brain. Co-infusion of an MRI contrast agent or radiolabeled albumin may not allow for precise therapeutic agent distribution analysis in the brain due to the differences in molecular weight and surface properties between each infusate. CED of maghemite magnetic nanoparticles has been depicted by MRI in a normal rat brain model. Perlstein et al., Neuro Oncol, 2008, 10:153-61.

The epidermal growth factor receptor variant III (EGFRvIII) is a tumor-specific mutation that is expressed in malignant gliomas and not in the normal brain. This mutation encodes a constitutively active tyrosine kinase that enhances tumorigenicity and accounts for radiation and chemotherapy resistance. The 801-bp in-frame deletion in the extracellular domain of the EGFR results in the fusion of normally distant EGFR gene and protein sequences. The 14-amino-acid fusion junction sequence has been chemically synthesized and used to create an anti-synthetic peptide antibody that is highly specific for the deletion mutant EGFR protein compared with the intact EGFR protein. Vaccination of the fusion junction peptide sequence has been shown to be efficacious immunotherapy in syngeneic murine models and in humans with two consecutive and one multi-institutional phase II trials. Sampson et al., Semin Immunol, 2008, 20:267-75.

Hadjipanayis et al. report EGFRvIII antibody-conjugated iron oxide nanoparticles for magnetic resonance imaging-guided convection-enhanced delivery and targeted therapy of glioblastoma. See Cancer Res, 2010, 70:6303-6312.

Platt et al., report a canine model of convection-enhanced delivery of cetuximab-conjugated iron-oxide nanoparticles monitored with magnetic resonance imaging. See Clin Neurosurg, 2012, 59:107-113. Using iron nanoparticles for cell-targeted imaging and effecting cell lysis is disclosed in PCT application publication WO2009/120702. However, there remains a need to provide improved compositions and methods.

Antibodies that bind to the extra-cellular domain of the human EGF receptor, such as Cetuximab, and other human monoclonal antibodies and proteins that bind to epidermal growth factor receptor (EGFR) are disclosed in U.S. Pat. Nos. 6,217,866, 7,247,301, 7,595,378, 7,589,180, 7,598,350, 7,696,320, 7,767,792, 7,887,805 and antibodies directed to the HER2 receptor U.S. Pat. No. 6,399,063.

The preceding is not intended to be an admission that any of the references cited herein are prior art.

### SUMMARY

The disclosure relates to metal particles compositions and uses related thereto. In certain embodiments, the disclosure relates to metal particles such as iron or iron oxide particles and their application in imaging and treating cancer.

In certain embodiment, the disclosure relates to methods of treating brain cancer comprising administering an effective amount of a pharmaceutical composition to a human subject in need thereof wherein the pharmaceutical compositions comprises an nanoparticle conjugated to a molecule that binds to the extra-cellular domain of the human EGF receptor.

In certain embodiments, the molecule is an antibody, antibody fragment, or antibody mimetic. In certain embodiments, the antibody is human/mouse chimeric monoclonal antibody wherein the antibody comprises human IgG1 heavy and kappa light chain constant regions produce by a mammalian cell culture. In certain embodiments, the antibody comprises the following CDR1, CDR2, and CDR3 sequences in a variable heavy region (SEQ ID NO: 1) Ser Tyr Trp Ile Glu, (SEQ ID NO: 2) Glu Ile Leu Pro Gly Ser Lys Lys Thr Asn Tyr Asn Glu Lys Phe Lys Gly, and (SEQ ID NO: 3) Tyr Tyr Tyr Arg Asn Asp Asp Tyr Gly Met Asp Tyr. In certain embodiments, the antibody comprises the following CDR1, CDR2, and CDR3 sequences in a variable light region (SEQ ID NO: 4) Ser Ala Ser Gln Asp Ile Arg Asn Tyr Leu Asn, (SEQ ID NO: 5) Tyr Thr Ser Thr Leu His Ser, and (SEQ ID NO: 6) Gln Gln Tyr Ser Lys Ile Pro Tyr Thr. In certain embodiments, the antibody comprises the following sequence in a variable heavy region (SEQ ID NO: 7) Gln Val Gln Leu Gln Gln Ser Gly Ala Glu Leu Met Lys Pro Gly Ala Ser Val Lys Ile Ser Cys Lys Ala Thr Gly Tyr Thr Phe Ser Ser Tyr Trp Ile Glu Trp Val Lys Gln Arg Pro Gly His Gly Leu Glu Trp Ile Gly Glu Ile Leu Pro Gly Ser Lys Lys Thr Asn Tyr Asn Glu Lys Phe Lys Gly Lys Ala Thr Phe Thr Ala Asp Thr Ser Ser Asn Thr Ala Tyr Met Gln Phe Ser Ser Leu Thr Ser Glu Asp Ser Ala Val Tyr Tyr Cys Ala Arg Tyr Tyr Tyr Arg Asn Asp Asp Tyr Gly Met Asp Tyr Trp Gly Gln Gly Thr Ser Val Thr Val Ser Ser. In certain embodiments, the antibody comprises the following sequence in the variable light region (SEQ ID NO: 8) Glu Ile His Met Thr Gln Thr Thr Ser Ser Leu Ser Ala Ser Leu Gly Asp Arg Val Thr Ile Ser Cys Ser Ala Ser Gln Asp Ile Arg Asn Tyr Leu Asn Trp Tyr Gln Gln Lys Pro Asp Gly Thr Val Lys Leu Leu Ile Tyr Tyr Thr Ser Thr Leu His Ser Gly Val Pro Ser Arg Phe Ser Gly Ser Gly Ser Gly Thr Asp Tyr Ser Leu Thr Ile Ser Asn Leu Glu Pro Glu Asp Ile Ala Thr Tyr Tyr Cys Gln Gln Tyr Ser Lys Ile Pro Tyr Thr Phe Thr Gly Gly Thr Lys Leu Glu Ile Lys Arg Ala Asp Ala Ala.

In certain embodiments, the disclosure contemplates methods where the pharmaceutical composition is administered intracranially such as by a catheter with an outer diameter of less than 1 mm. The particles are typically administered at greater than or about 0.01, 0.1, 0.25, 0.5 μg/min.

In certain embodiments, the subject is diagnosed with a brain tumor such as a glioma or glioblastoma.

In certain embodiments, the disclosure contemplates methods where the brain of the subject is exposed to an alternating magnetic field under conditions such that heat is generated about the area of the particles thus killing tumor cells.

In certain embodiments, the disclosure contemplates pharmaceutical compositions comprising a nanoparticle conjugated to a molecule that binds to the extra-cellular domain of the human EGF receptor. In certain embodiments, the pharmaceutical composition the form of an aqueous buffer, or phosphate buffer, pill, capsule, or tablet comprising a saccharide, polysaccharide, binder, preservative, filler, salt, or other a pharmaceutically acceptable excipient. Typically, the molecule that binds to the extra-cellular domain of the human EGF receptor is cetuximab.

In certain embodiments, the disclosure contemplates pharmaceutical compositions and uses described herein comprising particles such as iron oxide particles or iron particles with a substantially unoxidized core in combination with a second anticancer agent such as temozolamide, bevacizumab, procarbazine, lomustine, vincristine, gefitinib, erlotinib, docetaxel, cis-platin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, taxol, taxotere, etoposide, teniposide, amsacrine, topotecan, camptothecin, bortezomib, anagrelide, tamoxifen, toremifene, raloxifene, droloxifene, iodoxyfene, fulvestrant, bicalutamide, flutamide, nilutamide, cyproterone, goserelin, leuprorelin, buserelin, megestrol, anastrozole, letrozole, vorazole, exemestane, finasteride, marimastat, trastuzumab, cetuximab, dasatinib, imatinib, combretastatin, thalidomide, and/or lenalidomide or combinations thereof.

In certain embodiments, the disclosure relates to a particle comprising iron core coated with a hydrophobic layer and a hydrophilic layer. In certain embodiments, the particle further comprises a molecule that has affinity for a cell marker or receptor conjugated to the particle. Typically, the particle is a magnetic nanoparticle. In further embodiments, the hydrophilic layer comprises polyethylene glycol.

In certain embodiments, the disclosure relates to a particle comprising and iron oxide core or a substantially unoxidized iron core. In certain embodiments, the core is greater than 3, 4, or 5 nm and less than 10, 20, or 30 nm. In further embodiments, the core contains crystalline iron and an outside layer of crystalline iron oxide. In certain embodiments, the outside layer of iron oxide is greater than 1, 2, or 3 nm and less than 3 or 4 nm.

In certain embodiments, the molecule that has affinity for a cell marker or receptor may contain a polypeptide or the molecule that has affinity for a cell maker or receptor is an antibody, epitope, receptor, ligand, protein, or drug. The molecule that has affinity for a cell marker may be a tumor-specific antibody. In certain embodiments, the molecule that has affinity for a cell marker is an antibody to EGFR, EGFRvIII, VEGF, Erb1, Bcr-Abl, CD15, CD19, CD20, CD24, CD31, CD33, CD34, CD44, CD117, CD133, HER2, PDGF, CXCR4, TGF-beta, TGF-alpha, HIF-alpha, IGF-1, Sonic Hedgehog (SHH), Ras, TRAIL, Akt, p53, cMYC, nMYC, MDM2, CMET, PTEN, IL-13, IL-12, IL-4, IL-3 and BMP or an inhibitor of a receptor. In other embodiments, the tumor-specific antibody is avastin, rituxan, herceptin, pertuzumab, mylotarg, trastuzumab, panitumumab, or cetuximab.

In certain embodiments, the molecule that has affinity for a receptor is a cancer drug. In other embodiments, the molecule that has affinity for a receptor is a taxol, tarceva, rapamycin, or bryostatin.

In certain embodiments, the molecule that has affinity for a receptor is a molecule that inhibits one or more tyrosine kinases such as AATK, ABL1, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES, FGFR1, FGFR2, FGFR3, FGFR4, FGR, FLT1, FLT3, FLT4, FRK, FYN, GSG2, HCK, IGF1R, ILK, INSR, INSRR, IRAK4, ITK, JAK1, JAK2, JAK3, KDR, KIT, KSR1, LCK, LMTK2, LMTK3, LTK, LYN, MATK, MERTK, MET, MLTK, MST1R, MUSK, NPR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIE1, TNK1, TNK2, TNNI3K, TXK, TYK2, TYRO3, YES1, or ZAP70.

In certain embodiments, the molecule is lapatinib (Tykerb), sunitinib (Sutent), sorafenib (Nexavar), axitinib, pazopanib, cediranib, imatinib, gefitinib, dasatinib, erlotinib, nilotinib, mubritinib, E7080 with the IUPA name 4-[3-chloro-4-(cyclopropylcarbamoylamino)phenoxy]-7-methoxy-quinoline-6-carboxamide or BIBW 2992 with the IUPAC name N-[4-[(3-Chloro-4-fluorophenyl)amino]-7-[[(3S)-tetrahydro-3-furanyl]oxy]-6-quinazolinyl]-4(dimethylamino)-2-butenamide. In certain embodiments, the molecule that has affinity for a cell marker is a VEGF antibody such as bevacizumab (Avastin) and ranibizumab (Lucentis). In certain embodiments, the molecule that has affinity for a receptor is an aptamer such as pegaptanib.

In further embodiments, the target cells are cancer cells. Typically, the molecule that has affinity for a cell marker is an antibody to VEGF, PDGF receptor, EGF receptor, or truncated vIII EGF receptor.

In certain embodiments, the disclosure relates to targeting of GBM by local hyperthermia using composition and methods disclosed herein. Local hyperthermia can lead to induction of apoptosis, heat-shock protein release, and chemotherapy agent sensitivity of cancer cells with alternating fields (<1000 kHz) that are safe to normal cells.

In certain embodiments, the disclosure relates to a method of producing heat near a cell comprising, administering a nanoparticle comprising a substantially iron core conjugated to a molecule that has affinity for a cell marker or receptor and exposing the subject to an oscillating magnetic field under conditions such that the particle heats up.

In certain embodiments, the disclosure relates to a method for lysis of a target cell comprising, administering to a subject particles coated with a polysiloxane layer and a polyethylene glycol layer conjugated with a molecule that has affinity for a cell marker or receptor or that has an affinity for the target cells; and adjusting magnetic fields proximate the subject to cause cell lysis of the target cell. Typically, the magnetic field is an oscillating magnetic field and the particles are heated to at least 37° C. in vivo typically greater than 41° C. and less than 46° C. In certain embodiments, the subject is a human subject diagnosed with glioblastoma multiform and the particles are administered by intracerebral infusion under conditions such that intracranial pressure above normal is obtained.

In certain embodiments, the disclosure relates to methods for lysis of a target cell comprising, administering to a subject a particle with a molecule that has affinity for a cell marker or receptor or that has an affinity for the a target cell; and adjusting magnetic fields proximate the subject to cause cell lysis of the target cell, wherein the particles are administered by intracerebral infusion under conditions such that intracranial pressure above normal is obtained.

### BRIEF DESCRIPTION OF THE FIGURES

FIG. 1A shows data on the expression of EGFR and molecular profile of human glioma neurospheres. Western blot analysis of EGFR expression in human glioma neurospheres. Only one neurosphere culture from patient N08-30 is EGFRvIII positive. Expression of PTEN, phospho-Akt, and phospho-ERK44/42 was detected on the same Western blot. The lower panel shows that N08-74 and N08-30 neurosphere cultures maintain wtEGFR expression even at late passages (31 and 46, respectively). Normal human astrocytes, used as a control, express low levels of wtEGFR. Short and long exposures are shown.

FIG. 1B shows western blot analysis of expression of the stem cell marker CD133

FIG. 1C shows wester blot analysis of expression of stem cell markers nestin, Sox2, and Nanog. Total ERK was used as internal control. In all experiments, neurospheres were used in early passage.

FIG. 2A shows data on the physicochemical characterization and in vitro uptake of the cetuximab-IONPs. Illustration of amphiphilic triblock copolymer-coated IONPs conjugated to cetuximab.

FIG. 2B shows confirmation of conjugation of IONPs to cetuximab, EGFRvIII Ab, and a human IgG by mobility shift (black arrow) in 1% agarose gel.

FIG. 2C shows dynamic light scattering (DLS) and hydrodynamic diameter of IONPs, cetuximab-IONPs, and EGFRvIIIAb-IONPs.

FIG. 2D shows zeta potential of IONPs, EGFRvIIIAb-IONPs, and cetuximab-IONPs.

FIG. 2E shows transmission electron microscopy (TEM) studies of cell binding and internalization of cetuximab-IONPs into lysosomes of human glioma neurospheres N08-74 and N08-1002 (internalized nanoparticles are indicated by black arrows; magnification 10,000×).

FIG. 2F shows Prussian blue staining of IONPs, hIgG-IONPs, and cetuximab-IONPs internalized by human glioma neurospheres N08-30 (representative slides are shown). Neurospheres were allowed to reattach to cell culture dish after treatment in neurobasal media in the presence of 10% FCS. Nanoparticles are indicated by black arrows, magnification 40×. Cetuximab-IONPs showed maximal uptake.

FIG. 2G shows the effect of cetuximab-IONPs on phosphorylation of EGFR after activation with EGF. Human glioma neurospheres N08-30 and N08-1002 were starved for 24 hs, pretreated with cetuximab-IONPs, IONPs, hIgG-IONPs, or cetuximab for 3 hs, followed by activation with 100 ng/ml of EGF for 15 min. Western blotting with phospho-EGFR Y1068 antibody shows decreased activation of EGFR in the presence of cetuximab-IONPs.

FIG. 3A shows data on human glioma neurosphere and U87wtEGFR glioma cell line cytotoxicity after treatment by cetuximab-IONPs and quantification by an MTT assay. Neurospheres N08-74, N08-30, and N08-1002 (3×10⁴ cells per well), normal brain (NB, 5×103).

FIG. 3B shows data where U87wtEGFR cells (5×103) were treated with free IONPs (0.2 mg/ml), cetuximab-IONPs (0.2 mg/ml), control vehicle, and cetuximab alone (50 μg/ml). MTT assay was performed after 24, 48, and 72 hs for glioma neurospheres, 72 hs for normal brain cells, and 144 hs for the U87MGwtEGFR glioma cells. A significant decrease in cell survival was observed in glioma neurospheres treated with cetuximab-IONPs. No cytotoxicity was observed in normal brain cells after 72 hs. In all experiments, neurospheres and other cells were used in early passage. Absorbance was corrected by subtracting background and the statistical significance between experimental groups was tested by two-tailed Student's t-test, P<0.001; n=3.

FIG. 4A shows data on apoptosis in human glioma neurospheres and GSCs after treatment with cetuximab-IONPs. Neurospheres were treated with free IONPs, cetuximab-IONPs, control vehicle, and cetuximab alone and expression of apoptotic proteins were evaluated by Western blotting. Elevated levels of cleaved caspase 3 and cleaved PARP were found in neurospheres N08-74 and N08-30 after treatment with cetuximab-IONPs for 3 and

FIG. 4B shows data on apoptosis in human glioma neurospheres and GSCs after treatment with cetuximab-IONPs. Neurospheres were treated with free IONPs, cetuximab-IONPs, control vehicle, and cetuximab alone and expression of apoptotic proteins were evaluated by Western blotting. Elevated levels of cleaved caspase 3 and cleaved PARP were found in neurospheres N08-74 and N08-30 after treatment with cetuximab-IONPs for 14 hs. Treatment with cetuximab-IONPs was most effective in inducing cleavage of caspase 3 and PARP although some caspase 3 cleavage was also induced by free IONPs in N08-30. In neurospheres N08-1002, induction of caspase 3 and PARP cleavage was found after 3 h treatment with cetuximab-IONPs and cetuximab alone, both in the presence and absence of EGF and FGF

FIG. 4C shows treament with cetuximab-IONPs (but not the control conjugated antibody) increased cleavage of PARP in neurospheres N08-1002 whereas no cleavage was observed in human neuronal progenitor cells (NPCs)

FIG. 4D shows U87MG and U87MGwtEGFR human glioma cell lines were treated with free IONPs, cetuximab-IONPs, and cetuximab alone.

FIG. 4E shows Apoptosis, as indicated by activation of caspase 3 cleavage, was seen only in U87MGwtEGFR cell line treated with cetuximab-IONPs.

FIG. 5A shows data on the molecular profile and characterization of human GSCs.-FACS analysis.

FIG. 5B shows data on the molecular profile and characterization of human GSCs-Western blot of human GSCs (CD133-positive) and glioma CD133-negative neurospheres from patients N08-74 and N08-30.

FIG. 5C shows Hematoxylin and eosin (H&E) and immunohistochemistry staining of EGFRvIII and wtEGFR in orthotopic human malignant glioma xenografts generated in nude/athymic mice after intracranial implantation of N08-74 and N08-30 GSCs.

FIG. 5D shows an expression profile of selected proteins probed by Western blotting in human GSCs (CD133+) and CD133-negative neurospheres from patients N08-74, N08-30, and N08-1002.

FIG. 5E shows Prussian blue staining of IONPs and cetuximab-IONPs internalized by N08-74 GSCs (representative slides are shown). After 24 h treatment, neurospheres were allowed to reattach to cell culture dish in neurobasal media in the presence of 10% FCS and stained. Nanoparticles are indicated by black arrows, magnification 40×. Cetuximab-IONPs showed maximal uptake.

FIG. 5F shows Prussian blue staining of cetuximab-IONPs internalized by N08-30 GSCs. After 24 h incubation, GSCs were allowed to reattached to cell culture dish in neurobasal media in the presence of 10% FCS and stained (nanoparticles shown by black arrows, magnification 40×).

FIG. 5G shows Confocal microscopy of cetuximab-Cy5.5 and cetuximab-IONPs-Cy5.5 internalized by N08-74 GSCs. After 4 h treatment, GSCs were allowed to reattach to culture slides, fixed, and imaged using Zeiss LSM 510 Meta Confocal microscope. Cy5.5, pseudo-colored red; DAPI, pseudo-colored blue (magnification 100×).

FIG. 6A shows data on cytotoxicity of cetuximab-IONPs in human GSCs and glioma CD133-negative cells quantified by MTT assay. Human GSCs harvested from neurospheres N08-74, N08-30.

FIG. 6B shows data on cytotoxicity of cetuximab-IONPs in human GSCs and glioma CD133-negative cells quantified by MTT assay. Human GSCs harvested from neurospheres N08-1002 (3×104 cells per well) and glioma CD133-negative cells (3×104) were treated with free IONPs (0.2 mg/ml), cetuximab-IONPs (0.2 mg/ml), control vehicle, and cetuximab (50 μg/ml). MTT assay was performed after 24, 48, and 72 hs. A significant decrease in cell survival was found in all human GSCs treated with the cetuximab-IONPs for 72 hs; cetuximab-IONPs also decreased, to a lesser degree, the survival of human glioma CD133-negative cells. Absorbance was corrected by subtracting background and the statistical significance between experimental groups was tested by two-tailed Student's t-test P<0.001; n=3.

FIG. 6C shows a Western blot analysis of the CD133 status at the time of the MTT cytotoxicity assay.

FIG. 7A shows apoptosis studies, cell signaling in human GSCs, and animal survival studies after treatment with cetuximab-IONPs. GSCs and glioma CD133-negative neurospheres (5×10⁵ cells) from N08-74 treated with free IONPs, cetuximab-IONPs, and cetuximab for 3 hs and expression of cleaved caspase 3, caspase 3, cleaved PARP, and PARP was determined by Western blot analysis.

FIG. 7B shows apoptosis studies, cell signaling in human GSCs, and animal survival studies after treatment with cetuximab-IONPs. GSCs and glioma CD133-negative neurospheres (5×10⁵ cells) from N08-30 treated with free IONPs, cetuximab-IONPs, and cetuximab for 3 hs and expression of cleaved caspase 3, caspase 3, cleaved PARP, and PARP was determined by Western blot analysis

FIG. 7C is an Expression of cleaved caspase 9, caspase 9 after 3 h treatment in GSCs and glioma CD133-negative neurospheres (5×105 cells) from N08-30.

FIG. 7D are GSCs and CD133-negative cells from neurospheres N08-74 were treated with free IONPs (0.2 mg/ml), cetuximab-IONPs (0.2 mg/ml), and cetuximab (50 mg/ml) for 3 hs and analyzed by Western blotting with phospho-ERK44/42 and total ERK44/42 antibodies.

FIG. 7E shows Mice implanted with EGFR-expressing orthotopic human malignant glioma xenografts (U87wtEGFR) underwent convection-enhanced delivery (CED) with cetuximab-IONPs. Examples of T2 weighted MRI of mice brains showing a malignant glioma xenograft with a bright signal (white arrow) post tumor implantation (day 16) (a); MRI signal drop (black arrow) after cetuximab-IONPs CED (b). Tumor contrast enhancement after administration of gadolinium contrast agent in a control mouse (c) and a mouse treated with cetuximab-IONPs (d). White arrows indicate intracranial xenografts.

FIG. 7F shows Mice implanted with U87wtEGFR underwent CED with cetuximab-IONPs. T2 weighted MRI reveals the presence of cetuximab-IONPs (black arrows) and their distribution and dispersion on days 0, 8, 16, and 23, white arrow indicates intracranial xenograft. (G) Kaplan-Meier survival curve of athymic nude mice intracranially implanted with human U87MGwtEGFR cells and CED-treated with control, cetuximab, and cetuximab-IONPs. Statistical significance was estimated by log-rank method (P<0.001).

FIG. 8A shows data on the first canine EGFR-expressing spontaneous glioma patient CED-treated with cetuximab-IONPs. MRI scans with gadolinium contrast enhancement (gad) and T2WI were obtained pre-operative

FIG. 8B shows data on the first canine EGFR-expressing spontaneous glioma patient CED-treated with cetuximab-IONPs. MRI scans with gadolinium contrast enhancement (gad) and T2WI were obtained 24 hs and 7 days after partial tumor resection The large frontal mass is causing edema and mass effect on the surrounding brain. Intratumoral CED of cetuximab-IONPs was performed for 72 hs at a rate of 0.5 μl/min (2.16 ml total) after placement of two catheters. Residual tumor is seen postoperatively with evidence of T2 hypointense cetuximab-IONPs.

FIG. 8C are Postoperative MRI scans of first canine EGFR-expressing spontaneous glioma patient show a marked antitumor response 5 months after cetuximab-IONP CED treatment. Preoperative MRI (T2WI) shows large tumor (white arrow) and brain mass effect. Postoperative MRI (T2WI) at 6 weeks shows residual tumor (white arrow), with cetuximab-IONPs still present within tumor (black arrows).

FIG. 8D shows Hematoxylin and eosin (H&E) staining of canine brain glioma (magnification 20×, 40×). (E) Western blot analysis of canine brain tumor cells confirm wtEGFR positivity.

### DETAILED DISCUSSION

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

The term “subject” refers to any animal, preferably a human patient, livestock, or domestic pet.

As used herein, the terms “treat” and “treating” are not limited to the case where the subject (e.g. patient) is cured and the disease is eradicated. Rather, the present disclosure also contemplates treatment that merely reduces symptoms, and/or delays disease progression.

The terms “iron particle” and “iron core” refer to a material that contains a substantial amount of the iron atoms. The iron may take the form of a salt, an iron oxide, or a solid lattice of iron atoms, i.e., crystalline iron. Crystalline iron will oxidize, e.g., rust, when exposed to atmospheric oxygen.

As used herein “cancer” refers any of various cellular diseases with malignant neoplasms characterized by the proliferation of cells. Most cancers form a tumor but some, like leukemia, do not. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed. It may also be identified by a change in relevant biomarker or gene expression profile, such as HER2 for breast cancer.

### Targeted Therapy of Glioma Stem Cells and Tumor Non-Stem Cells Using EGFR Antibody-Conjugated Iron Oxide Nanoparticles

The wild-type (wt) EGFR is frequently overexpressed in ˜60% of glioblastoma (GBM), whereas EGFRvIII expression is detected in up to 30% of GBM tumors. The Cancer Genome A high level of EGFR expression indicates poor prognosis in GBM patients. EGFR may be used for targeting Glioma stem cells (GSCs). Cetuximab is a 152 kDa chimeric monoclonal antibody of the immunoglobulin G1 subclass that binds to the extracellular domain of the human EGFR. This antibody recognizes both wtEGFR and EGFRvIII mutant with similar affinity, inhibiting both ligand binding to wtEGFR and autophosphorylation of EGFRvIII.

The therapeutic targeting effect of cetuximab-IONPs against GSCs and glioma tumor non-stem cells was to investigate. In addition, therapeutic targeting in vivo was assessed using CED in two experimental models: human intracranial glioma xenografts in a rodent model and a spontaneous canine glioma. Compared to cetuximab alone, cetuximab-IONPs were more cytotoxic in vitro and induced apoptosis in human GSCs (CD133-positive cells), CD133-negative glioma cells, as well as unsorted human glioma neurospheres. Greater uptake of cetuximab-IONPs was found in GSCs and glioma cells in comparison to cetuximab alone Inhibition of EGFR signaling was achieved after treatment of human glioma neurospheres and GSCs with the cetuximab-IONPs. The targeted therapy of cetuximab-IONPs and CED in vivo revealed a significant therapeutic effect in an orthotopic mouse model of glioma. Furthermore, a canine patient with a large spontaneous glioma, had a marked antitumor response after CED treatment with cetuximab-IONPs.

IONPs bioconjugated to the chimeric monoclonal EGFR antibody cetuximab have a therapeutic effect against malignant gliomas, the most lethal form of brain cancer. In side-by-side comparisons, the cetuximab-IONPs were more effective than cetuximab alone and offered a significantly increased tumor cell toxicity in vitro against human glioma neurospheres, GSCs, and glioma CD133-negative cells expressing various levels of wtEGFR. The significant in vitro effect was also observed in human glioma neurospheres, GSCs, and CD133-negative tumor cells which also expressed the EGFRvIII deletion mutant. No significant toxicity was found with normal brain.

The cetuximab-IONPs bind to both the wtEGFR and the EGFRvIII deletion mutant, are internalized by the tumor cells, and that conjugation with IONPs enhances internalization. Although it is not intended that embodiments of the disclosure be limited by any particular mechanism, it is believe when internalized cetuximab-IONPs accumulate, a pro-apoptotic signal is triggered through cleavage of caspase 9, followed by cleavage of caspase 3 that eventually causes cancer cell death.

Data suggest that cetuximab conjugation to IONPs amplifies the biological effect of cetuximab and cetuximab-IONPs are effective against GBM malignant glioma cells with varying levels of EGFR expression. Cetuximab-IONPs, but not cetuximab alone, specifically downregulated VEGF secretion in tumor cells is at variance with a previous report that cetuximab decreased VEGF expression in GBM cell lines (Eller et al., Neurosurgery, 2002, 51:1005-1013; discussion 1013-1004). This is most likely due to a different concentration of cetuximab used in comparison to our studies but it does suggest the higher biological potency of the EGFR-antibody conjugated IONPs.

The data presented does show that cetuximab-IONPs have a significant therapeutic effect with in vivo glioma models. Cetuximab-IONP CED permitted direct imaging by MRI, revealing intratumoral and peritumoral localization of the nanoparticles in a rodent malignant glioma model with further distribution of IONPs over weeks. Treatment with cetuximab-IONPs promoted apoptosis of GSCs in vitro, exerted a significant therapeutic effect in vivo in a malignant glioma mouse model after CED, and showed a dramatic antitumor effect in a canine spontaneous glioma model after a single CED treatment. Cetuximab-IONPs are safe, can be visualized on standard T2 weighted MRI, are retained in brain tumors for many weeks with no toxicity to the surrounding brain, and have a significant anti-tumor effect in vitro and in vivo. GSCs can be targeted with cetuximab-conjugated IONPs. Human clinical trials for patients with GBM is contemplated.

### Antibodies and Antibody Mimetics

In certain embodiments, the disclosure contemplates molecules that target cancer markers disclosed herein, such as EGFR, that are antibodies or fragments or chimera, antibody mimetics, or aptamers.

Numerous methods known to those skilled in the art are available for obtaining antibodies or antigen-binding fragments thereof. For example, antibodies can be produced using recombinant DNA methods (U.S. Pat. No. 4,816,567). Monoclonal antibodies may also be produced by generation of hybridomas in accordance with known methods. Hybridomas formed in this manner are then screened using standard methods, such as enzyme-linked immunosorbent assay (ELISA) and surface plasmon resonance analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

The modular structure of antibodies makes it possible to remove constant domains in order to reduce size and still retain antigen binding specificity. Engineered antibody fragments allow one to create antibody libraries. A single-chain antibody (scFv) is an antibody fragment where the variable domains of the heavy (V_{H}) and light chains (V_{L}) are combined with a flexible polypeptide linker. The scFv and Fab fragments are both monovalent binders but they can be engineered into multivalent binders to gain avidity effects. One exemplary method of making antibodies and fragments includes screening protein expression libraries, e.g., phage or ribosome display libraries. Phage display is described, for example, in U.S. Pat. No. 5,223,409.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. U.S. Pat. No. 7,064,244.

Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Computational methods may be utilized to generate fully human mAbs from nonhuman variable regions using information from the human germline repertoire. See Pat. Nos. 8,314,213, 7,930,107, 7,317,091, Bernett et al., entitled, “Engineering fully human monoclonal antibodies from murine variable regions,” J Mol Biol. 2010, 396(5):1474-90.

In certain embodiments, a humanized antibody is optimized by the introduction of conservative substitutions, consensus sequence substitutions, germline substitutions and/or backmutations. An antibody or fragment thereof may also be modified by specific deletion of human T cell epitopes or “deimmunization” by the methods disclosed in U.S. Pat. No. 7,125,689 and U.S. Pat. No. 7,264,806. Briefly, the heavy and light chain variable domains of an antibody can be analyzed for peptides that bind to MHC Class II; these peptides represent potential T-cell epitopes. For detection of potential T-cell epitopes, a computer modeling approach termed “peptide threading” can be applied, and in addition a database of human MHC class II binding peptides can be searched for motifs present in the VH and VL sequences. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. The V BASE directory provides a comprehensive directory of human immunoglobulin variable region sequences. These sequences can be used as a source of human sequence, e.g., for framework regions and CDRs. Consensus human framework regions can also be used, e.g., as described in U.S. Pat. No. 6,300,064.

Antibody mimetics or engineered affinity proteins are polypeptide based targeting moieties that can specifically bind to targets but are not specifically derived from antibody VH and VL sequences. Typically, a protein motif is recognized to be conserved among a number of proteins. One can artificially create libraries of these polypeptides with amino acid diversity and screen them for binding to targets through phage, yeast, bacterial display systems, cell-free selections, and non-display systems. See Gronwall & Stahl, J Biotechnology, 2009, 140(3-4), 254-269, hereby incorporated by reference in its entirety. Antibody mimetics include affibody molecules, affilins, affitins, anticalins, avimers, darpins, fynomers, kunitz domain peptides, and monobodies.

Affibody molecules are based on a protein domain derived from staphylococcal protein A (SPA). SPA protein domain denoted Z consists of three α-helices forming a bundle structure and binds the Fc protion of human IgG1. A combinatorial library may be created by varying surface exposed residues involved in the native interaction with Fc. Affinity proteins can be isolated from the library by phage display selection technology.

Monobodies, sometimes referred to as adnectins, are antibody mimics based on the scaffold of the fibronectin type III domain (FN3). See Koide et al., Methods Mol. Biol. 2007, 352: 95-109, hereby incorporated by reference in its entirety. FN3 is a 10 kDa, β-sheet domain, that resembles the VH domain of an antibody with three distinct CDR-like loops, but lack disulfide bonds. FN3 libraries with randomized loops have successfully generated binders via phage display (M13 gene 3, gene 8; T7), mRNA display, yeast display and yeast two-hybrid systems. See Bloom & Calabro, Drug Discovery Today, 2009, 14(19-20):949-955, hereby incorporated by reference in its entirety.

Anticalins, sometimes referred to as lipocalins, are a group of proteins characterized by a structurally conserved rigid f3-barrel structure and four flexible loops. The variable loop structures form an entry to a ligand-binding cavity. Several libraries have been constructed based on natural human lipocalins, i.e., ApoD, NGAL, and Tlc. Anticalins have been generated for targeting the cytotoxic T-lymphocyte antigen-4 (CTLA-4). See Skerra, FEBS J., 275 (2008), pp. 2677-2683, and Binder et al., J Mol Biol., 2010, 400(4):783-802, both hereby incorporated by reference in their entirety.

The ankyrin repeat (AR) protein is composed repeat domains consisting of a β-turn followed by two α-helices. Natural ankyrin repeat proteins normally consist of four to six repeats. The ankyrin repeats form a basis for darpins (designed ankyrin repeat protein) which is a scaffold comprised of repeats of an artificial consensus ankyrin repeat domain. Combinatorial libraries have been created by randomizing residues in one repeat domain. Different numbers of the generated repeat modules can be connected together and flanked on each side by a capping repeat. The darpin libraries are typically denoted N×C, where N stands for the N-terminal capping unit, C stands for the C-terminal capping domain and x for the number of library repeat domains, typically between two to four. See Zahnd et al., J. Mol. Biol., 2007, 369:1015-1028, hereby incorporated by reference in its entirety.

Aptamers refer to affinity binding molecules identified from random proteins or nucleic acids libraries. Peptide aptamers have been selected from random loop libraries displayed on TrxA. See Borghouts et al., Expert Opin. Biol. Ther., 2005, 5:783-797, hereby incorporated by reference in its entirety. SELEX (“Systematic Evolution of Ligands by Exponential Enrichment”) is a combinatorial chemistry technique for producing oligonucleotides of either single-stranded DNA or RNA that specifically bind to a target. Standard details on generating nucleic acid aptamers can be found in U.S. Pat. No. 5,475,096, and U.S. Pat. No. 5,270,163. The SELEX process provides a class of products which are referred to as nucleic acid ligands or aptamers, which has the property of binding specifically to a desired target compound or molecule. Each SELEX-identified nucleic acid ligand is a specific ligand of a given target compound or molecule. The SELEX process is based on the fact that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

### EGFR Deletion Mutant EGFRvIII

Anti-EGFR antibodies and small-molecule EGFR tyrosine kinase inhibitors have entered the clinical setting as systemic administered agent. Traditionally, these agents have shown modest efficacy in patients with GBM tumors. The development of new agents that can target the EGFR deletion mutant EGFRvIII can permit direct targeting of glioblastoma cells while sparing the normal brain. Furthermore, the targeting of GSCs and/or GSC-specific molecules (e.g., CD133) forms the basis of more effective treatments against glioblastoma tumors and prevention of relapse. In certain embodiments, the disclosure relates to anti-EGFR antibodies conjugated to particles and uses disclosed herein. In other embodiments, the disclosure relates to EGFR tyrosine kinase inhibitors conjugated to particles and uses disclosed herein.

In certain embodiments, the disclosure relates to the use of iron nanoparticles conjugated to an EGFRvIII deletion mutant antibody for MRI-assisted CED and targeted therapy of human glioblastoma. The conjugated EGFRvIIIAb-iron nanoparticles permit MRI contrast enhancement or “darkening” of U87ΔEGFRvIII cells with T2-weighted MRI. The EGFRvIIIAb-IONPs (iron oxide nanoparticles) caused a significant decrease in glioblastoma cell survival, and a greater antitumor effect was found after treatment of EGFRvIII-expressing glioblastoma cells with EGFRvIIIAb-IONPs in comparison with human glioblastoma cells, which did not express the EGFR.

Although is not intended that embodiments of the disclosure be limited by any particular mechanism, the pronounced antitumor effect of free IONPs in glioblastoma cells is likely due to the nonspecific uptake of the nanoparticles by the tumor cells. Uptake of IONPs by glioma cells has been shown in culture and in vivo. The influence of surface functionalization has been shown to enhance the internalization of magnetic nanoparticles in cancer cells. Villanueva et al., Nanotechnology, 2009, 20:115103. It is contemplated that certain iron nanoparticles disclosed herein may promote uptake within glioblastoma cells and result in cell toxicity. Thus in certain embodiments, the disclosure relates to treating cancer by administering compositions disclosed herein.

No significant toxicity was found with IONP treatment in human astrocytes or in animals after intracerebral administration. No toxicity to human astrocytes and a significant killing effect of both free IONPs and EGFRvIIIAb-IONPs form the basis of targeted therapy of GBM cells in the brain. Apoptosis was determined to be a mode of cell death after treatment of glioblastoma cells and neurospheres by the conjugate EGFRvIIIAb-IONP. Apoptosis was found after treatment of GSC-containing neurospheres harvested from a glioblastoma patient with elevated expression of EGFRvIII and the GSC marker CD133. A significant antitumor effect was found both in vitro and in our animal survival studies. Mechanistic studies suggest that EGFR downstream signaling may be affected by IONPs with less EGFR phosphorylation after glioblastoma cell treatment with EGFRvIIIAb-IONPs. Furthermore, IONPs resulted in less phosphorylation of Akt and ERK in glioblastoma cells overexpressing the EGFR.

CED of IONPs in a mouse glioma model results in MRI contrast of the nanoparticles and effective intratumoral and peritumoral distribution of nanoparticles in the brain. A significant therapeutic effect was found after CED of both IONPs and EGFRvIIIAb-IONPs in mice. Dispersion of the nanoparticles over days, after the infusion has finished, may potentially target infiltrating tumor cells outside the tumor mass that are potentially responsible for tumor recurrence and the demise of patients. Use of bioconjugated magnetic nanoparticles may permit the advancement of CED in the treatment of malignant gliomas due to their sensitive imaging qualities on standard T2-weighted MRI and therapeutic effects. Better targeting of infiltrative glioblastoma tumors by MRI-guided CED of magnetic nanoparticles may provide more effective treatment of these devastating brain tumors.

### Convection-Enhanced Delivery (CED)

In certain embodiments, the disclosure relates to administering compositions disclosed herein by convection-enhanced delivery. CED is a method of administrating compositions by direct infusion into the brain interstitial spaces utilizing a fluid pressure gradient after catheter placement. IONPs produce an MRI T2 signal drop in the rodent brain after CED permitting direct visualization of nanoparticle distribution. Visualizing distribution of infused composition is desirable to ensure accurate delivery into target sites and provides feedback on catheter placement and control of the administered composition. Leakage of infusate into the ventricular or subarachnoid spaces is undesirable. Homogenous and targeted distribution of IONPs is evidenced by MRI after CED.

A barrier in the use of certain nanoparticles for brain tumor applications is the difficulty of contacting the nanoparticles with intracranial tumors. Systemic delivery has the disadvantages due to the nonspecific nanoparticle uptake by the reticuloendothelial system and problems in penetrating the blood-brain barrier (BBB). Convection-enhanced delivery (CED) provides bulk flow of fluid in the brain by a pressure gradient that bypasses the BBB. Therapeutic agents can be delivered into the brain by CED without toxicity to normal tissue and organs commonly associated with systemic delivery. The use of CED allows for therapeutic targeting of infiltrating cancer cells, a major cause for brain tumor recurrence after surgery.

In certain embodiments, the disclosure relates to the use of iron nanoparticles conjugated to an anti-synthetic peptide antibody (EGFRvIIIAb) specific to the deletion-mutant EGFR for image-guided CED in a subject with a glioma. The EGFRvIIIAb-iron nanoparticle is used as a MRI contrast enhancement of human glioblastoma cells producing antitumor effect both in vitro and in vivo after CED.

### Magnetic Metal Particles

In certain embodiments, the disclosure relates to a magnetic nanoparticle that can provide cancer cell detection by magnetic resonance imaging (MRI) contrast enhancement as well as therapy by cancer cell-targeted delivery of therapeutic agents (antibodies, drugs, and small-molecule inhibitors) or by local hyperthermia generated by absorbing energy from an alternating magnetic field. Iron nanoparticles, typically in the size range of 10 to 25 nm, have unique magnetic properties, generating significant transverse T2 relaxation time shortening and susceptibility effects resulting in strong T2-weighted contrast on MRI. Desirable iron nanoparticles evade the immune system and target cancer cells for destruction while simultaneously providing MRI contrast. Desirable iron nanoparticles are biodegradable and have low toxicity. In certain embodiments, the disclosure relates to the use of iron nanoparticles for drug delivery schemes, magnetic cell separation and cell targeting, magnetic resonance imaging (MRI) contrast enhancement, and hyperthermia treatment of cancer.

In certain embodiments, the disclosure relates to cell-specific imaging for detection and treatment monitoring for the therapy of various cancers including central nervous system (CNS) tumors.

Increasing the magnetization of nanoparticles can be accomplished by utilizing elemental iron (Fe) but limitations of pure Fe nanoparticles include their potential oxidation and instability in aqueous solution. Issues of biocompatibility, stability, and size restriction are addressed by coating the nanoparticles with an iron oxide (Fe—O) shell. In certain embodiments, the disclosure relates to iron nanoparticles with certain size distribution and Fe/Fe3O4 core/shell morphology. These compositions may be synthesized by the thermal decomposition of iron carbonyls. Coating of the iron nanoparticles may be achieved with a diblock copolymer or amphiphilic multidentate ligands for oxidation protection, surface functionalization, and EGFR antibody bioconjugation. The targeting and MRI contrast effect of the bioconjugated iron nanoparticles may be determined on human GBM cells (GSC-containing neurospheres and GBM cell lines). In certain embodiments, the disclosure relates to local hyperthermia generation and the antitumor effect on GBM cells after treatment with bioconjugated iron nanoparticles by applying alternating current magnetic fields (100-400 kHz).

In certain embodiments of the disclosure, it is contemplated that magnetically stronger nanoparticles with a small size but high mass-magnetization values provide greater MRI contrast enhancement as well as other functions (magnetic targeting and hyperthermia) for cancer nanotechnology applications. Increasing the magnetization of nanoparticles may be accomplished by utilizing elemental iron (Fe). Pure Fe nanoparticles may oxidize resulting in instability in aqueous solution.

In certain embodiments, the disclosure relates to the coating or creating an elemental iron nanoparticle with an iron oxide (Fe—O) shell providing biocompatible polymer coating. The addition of certain coatings provides greater water solubility and permit surface functionalization for conjugating bioactive ligands. In certain embodiments, the disclosure relates to polymer coated elemental-based nanoparticles, optionally contain an iron oxide coat, that can be bioconjugated with GBM specific antibodies and their use in tumor cell detection, treatment, and therapeutic follow-up monitoring.

The elemental iron based nanoparticles achieve higher magnetization (70 emu g-1) than the IONPs without the introduction of other metal elements that may be biologically toxic. A thin shell layer of iron-oxide (Fe3O4) surrounding the iron core provides an oxidative protective layer to the magnetic nanoparticles and permits their retention of high magnetic properties, stability, and biocompatibility. Hadjipanayis et al., Small. 2008; 4:1925-1929. The elemental iron particles may be synthesized chemically, e.g., by the thermal decomposition of iron pentacarbonyl in the presence of organic solvents and surfactants. See Peng et al., J. Am. Chem. Soc. 128, 10676 (2006). Disclosed herein is a core and shell in highly crystalline state without adding any oxidizing agent. In certain embodiments, the disclosure relates to crystalline iron nanoparticles produced without the use of an oxidizing agent.

Conventional bright field transmission electron microscopy (TEM) studies revealed a core-shell structure in these particles. The measured lattice spacing of the core corresponds to the (110) lattice planes of alpha iron and that of the shell to the (311) planes of the spinel iron oxide. The Fe core is single crystal; however, the oxide shell is composed of small crystallites which are oriented randomly. When exposed to air, the particles surface got oxidized making a thick oxide shell around the core. Peng et al. reported the formation of amorphous iron oxide shell at a reaction temperature of 180° C. J. Am. Chem. Soc. 128, 10676 (2006). Metallic Fe nanoparticles coated by an amorphous iron oxide shell are not protected from deep oxidation while the crystalline Fe3O4 shell offers more robust protection to the metallic core. The crystalline Fe3O4 shell gives better stability to these particles toward deep oxidation and makes these materials more appropriate for biomedical applications.

In order to investigate the effect of injection temperature on the particle properties, several samples were synthesized at temperature from 200 to 315° C. The reaction mixture was cooled down to room temperature immediately after the injection of Fe(CO)₅. It has been observed that the average particle size increases with the injection temperature, and the size distribution gets broader. For example, 8, 12, and 17 nm average diameter particles can be obtained when the injection temperature is 225, 250, and 295° C., and this can be attributed to the increased growth rate of nucleus with temperature. However, the thickness of oxide shell remains unchanged. The room temperature magnetization also increases with injection temperature from 80 to 140 emu/g presumably because of the increasing average particle size. These particles showed a strong ferromagnetic behavior with coercivity above 300 Oe. This higher value of coercivity is attributed to the noncollinear surface spin structures between the soft bcc iron and the harder fcc iron oxide phase. In certain embodiments, the disclosure relates to a crystalline iron core particle size of 5-12 nm and a shell thickness 2-4 nm.

Keeping the injection and refluxing temperature the same, a temperature of 300° C. was chosen to study the effect of refluxing time on these particles. Along with the characteristic reflections from alpha iron at 44.7°, peaks at around 39.5°, 41°, and 43.6° are becoming more and more pronounced with increased refluxing time. These peaks are characteristic reflections of iron carbide Fe₃C and Fe₅C₂. Iron/iron-carbide nanocomposite particles are more stable and oxidation resistant. In certain embodiments, the disclosure relates to iron/iron-carbide nanocomposite particles.

In certain embodiments, the disclosure relates to the use of polysiloxane containing diblock copolymer that can be used to transform and stabilize iron nanoparticles into aqueous medium. Chen et al. ACS Appl. Mater. and Interfaces, 2009, 1:2134-2140. The silanol groups present on the polymer surface are readily available for reacting with a variety of functional groups allowing for the bioconjugation of cancer cell targeting and affinity ligands.

In certain embodiments, the disclosure relates to the coating of iron nanoparticles with amphiphilic multidentate ligands. Kairdolf et al., J Am Chem Soc. 2008; 130:12866-12867.

Multidentate polymer ligands contain aliphatic chains and carboxylic acid functional groups and are found to act as both a nanoparticle ligand and a surface stabilizer.

Typically, one synthesizes the amphiphilic polymer by coupling poly(acrylic) acid and dodecylamine. Typically half of the carboxylic acid functional groups are modified with a moiety with a hydrocarbon/aliphatic chain. One may synthesizes the iron cores using multidentate polymer ligands. One mixes the amphiphlic polymer with PEG and an iron salt at elevated temperatures.

In certain embodiments, the disclosure relates to iron nanoparticles coated with polysiloxane polymers and amphiphilic polymers conjugated to molecule that has affinity for a specific polypeptide, typically a EGFRvIII antibody or cetuximab.

### Glioblastoma Multiforme (GBM)

Glioblastoma is the most common and most aggressive type of primary brain tumor in humans, involving glial cells and accounting for 52% of all parenchymal brain tumor cases and 20% of all intracranial tumors. Despite being the most prevalent form of primary brain tumor, GBMs occur in only 2-3 cases per 100,000 people in Europe and North America.

According to the WHO classification of the tumors of the central nervous system, the standard name for this brain tumor is “glioblastoma”; it presents two variants: giant cell glioblastoma and gliosarcoma. Glioblastomas are also an important brain tumor of the canine, and research is ongoing to use this as a model for developing treatments in humans.

In certain embodiments the disclosure relates to the use of compositions and method disclosed herein in combination with therapies described herein. Treatment can involve chemotherapy, radiation, radiosurgery, corticosteroids, antiangiogenic therapy, and surgery. Glioblastoma has a very poor prognosis, despite multimodality treatment consisting of open craniotomy with surgical resection of as much of the tumor as possible, followed by concurrent or sequential chemoradiotherapy, antiangiogenic therapy with bevacizumab, gamma knife radiosurgery, and symptomatic care with corticosteroids. Other than the brainstem gliomas, it has the worst prognosis of any CNS malignancy.

Although common symptoms of the disease include seizure, nausea and vomiting, headache, and hemiparesis, the single most prevalent symptom is a progressive memory, personality, or neurological deficit due to temporal and frontal lobe involvement. The kind of symptoms produced depends highly on the location of the tumor, more so than on its pathological properties. The tumor can start producing symptoms quickly, but occasionally is an asymptomatic condition until it reaches an enormous size.

It is very difficult to treat glioblastoma due to several complicating factors: The tumor cells are very resistant to other conventional therapies, the brain is susceptible to damage due to conventional therapy, the brain has a very limited capacity to repair itself and many drugs cannot cross the blood-brain barrier to act on the tumor. Treatment of primary brain tumors and brain metastases consists of both symptomatic and palliative therapies. Supportive treatment focuses on relieving symptoms and improving the patient's neurologic function. The primary supportive agents are anticonvulsants and corticosteroids.

Many of patients with glioblastoma undergo anticonvulsant treatments. Those receiving phenytoin concurrent with radiation may have serious skin reactions such as erythema multiforme and Stevens-Johnson syndrome. Dexamethasone given 4 to 10 mg every 4 to 6 h, can reduce peritumoral edema (through rearrangement of the blood-brain barrier) diminishing mass effect and lowering intracranial pressure with a decrease in headache or drowsiness.

Palliative treatment usually is conducted to improve quality of life and to achieve a longer survival time. It includes surgery, radiation therapy, and chemotherapy. A maximally feasible resection with maximal tumor-free margins is usually performed along with external beam radiation and chemotherapy. Gross total resection of tumor is associated with a better prognosis. Surgery is the first stage of treatment of glioblastoma.

The chances of near-complete initial removal of the tumor can be greatly increased if the surgery is guided by a fluorescent dye known as 5-aminolevulinic acid. On average, radiotherapy after surgery can reduce the tumor size. Whole brain radiotherapy does not improve when compared to the more precise and targeted three-dimensional conformal radiotherapy. A total radiation dose of 60-65 Gy is typical for treatment. Boron neutron capture therapy has been tested as an alternative treatment for glioblastoma multiforme.

Treatment of glioblastoma includes chemotherapy during and after radiotherapy. On average, chemotherapy after surgery and radiotherapy can initially reduce the tumor size. The use of temozolomide both during radiotherapy and for six months post radiotherapy results in a significant increase in median survival with minimal additional toxicity. This treatment regime is now standard for most cases of glioblastoma where the patient is not enrolled in a clinical trial. Temozolomide seems to work by sensitizing the tumor cells to radiation. The U.S. Food and Drug Administration approved Avastin (bevacizumab) to treat patients with glioblastoma at progression after standard therapy.

Patients most fear the adverse effects of systemic chemotherapy when undergoing treatment for cancer. Nausea and vomiting are the most common and severe side effects. Other adverse side effects include cytopenia, infection, cachexia, mucositis in patients receiving high doses of chemotherapy with bone marrow rescue or radiation therapy; alopecia (hair loss); cutaneous complications such as pruritis, urticaria, and angioedema; neurological complications; pulmonary and cardiac complications in patients receiving radiation or chemotherapy; and reproductive and endocrine complications.

Chemotherapy-induced side effects significantly impact the quality of life of the patient and may dramatically influence patient compliance with treatment. Additionally, adverse side effects associated with chemotherapeutic agents are generally the major dose-limiting toxicity (DLT) in the administration of these drugs. For example, mucositis, is one of the major dose limiting toxicity for several anticancer agents, including the antimetabolite cytotoxic agents 5-FU, methotrexate, and antitumor antibiotics, such as doxorubicin. Many of these chemotherapy-induced side effects if severe, may lead to hospitalization, or require treatment with analgesics for the treatment of pain. The adverse side effects induced by chemotherapeutic agents and radiation therapy have become of major importance to the clinical management of cancer patients.

In certain embodiments, the disclosure relates to administering compositions disclosed herein, typically a nanoparticle with an iron core to a subject suffering from or at risk of a CNS tumor. Tumors of the CNS include but are not limited to cancers such as neuroblastoma and glioblastoma. Other types of central nervous system tumors include: Neuroepithelial Tumors of the CNS, Astrocytic tumors [glial tumors—categories I-V, below—may also be subclassified as invasive or non-invasive, although this is not formally part of the WHO system, the non-invasive tumor types are indicated below. Categories in italics are also not recognized by the new WHO classification system, but are in common use.]; Astrocytoma (WHO grade II); protoplasmic, gemistocytic, fibrillary, Anaplastic (malignant) astrocytoma (WHO grade III); hemispheric; diencephalic; optic; brain stem; cerebellar; Glioblastoma multiforme (WHO grade IV); giant cell glioblastoma, gliosarcoma; Pilocytic astrocytoma [non-invasive, WHO grade I]; hemispheric; diencephalic; optic; brain stem; cerebellar; Subependymal giant cell astrocytoma [non-invasive, WHO grade I]; Pleomorphic xanthoastrocytoma [non-invasive, WHO grade I]; Oligodendroglial tumors; Oligodendroglioma (WHO grade II); Anaplastic (malignant) oligodendroglioma (WHO grade III); Ependymal cell tumors; Ependymoma (WHO grade II); cellular, papillary, epithelial, clear cell; Anaplastic ependymoma (WHO grade III); Myxopapillary ependymoma; Subependymoma (WHO grade I); Anaplastic (malignant) oligoastrocytoma (WHO grade III); Neuroepithelial tumors of uncertain origin; Polar spongioblastoma (WHO grade IV); Astroblastoma (WHO grade IV); Gliomatosis cerebri (WHO grade IV); Tumors of the choroid plexus; Choroid plexus papilloma; Choroid plexus carcinoma (anaplastic choroid plexus papilloma); Neuronal and mixed neuronal-glial tumors; Gangliocytoma; Dysplastic gangliocytoma of cerebellum (Lhermitte-Duclos); Ganglioglioma; Anaplastic (malignant) ganglioglioma; Desmoplastic infantile ganglioglioma; desmoplastic infantile astrocytoma; Central neurocytoma; Dysembryoplastic neuroepithelial tumor; Olfactory neuroblastoma (esthesioneuroblastoma); olfactory neuroepithelioma; Pineal Parenchyma Tumors; Pineocytoma; Pineoblastoma; Mixed pineocytoma/pineoblastoma; Tumors with neuroblastic or glioblastic elements (embryonal tumors); Medulloepithelioma; Primitive neuroectodermal tumors with multipotent differentiation; medulloblastoma; medullomyoblastoma, melanocytic medulloblastoma, desmoplastic medulloblastoma; cerebral primitive neuroectodermal tumor; Neuroblastoma; ganglioneuroblastoma; Retinoblastoma; Ependymoblastoma; CNS Neoplasms; Tumors of the Sellar Region; Pituitary adenoma; Pituitary carcinoma; Craniopharyngioma; Hematopoietic tumors; Primary malignant lymphomas; Plasmacytoma; Granulocytic sarcoma; Germ Cell Tumors; Germinoma; Embryonal carcinoma; Yolk sac tumor (endodermal sinus tumor); Choriocarcinoma; Teratoma; Tumors of the Meninges; Meningioma; meningothelial, fibrous (fibroblastic), transitional (mixed), psammomatous, angiomatous, microcystic, secretory, clear cell, chordoid, lymphoplasmacyte-rich, and metaplastic subtypes; Atypical meningioma; Anaplastic (malignant) meningioma; Non-menigothelial tumors of the meninges; Benign Mesenchymal; osteocartilaginous tumors; lipoma; fibrous histiocytoma; Malignant Mesenchymal; chondrosarcoma; hemangiopericytoma; rhabdomyosarcoma; meningeal sarcomatosis; Primary Melanocytic Lesions; diffuse melanosis; melanocytoma; maliganant melanoma; Hemopoietic Neoplasms; malignant lymphoma; plasmactoma; granulocytic sarcoma; Tumors of Uncertain Histogenesis such as hemangioblastoma (capillary hemangioblastoma); Tumors of Cranial and Spinal Nerves such as Schwannoma (neurinoma, neurilemoma); cellular, plexiform, and melanotic subtypes; Neurofibroma; circumscribed (solitary) neurofibroma; plexiform; neurofibroma; Malignant peripheral nerve sheath tumor (Malignant schwannoma); epithelioid; divergent mesenchymal or epithelial differentiation; and melanotic.

### EXPERIMENTAL

### EGFR Expression in Malignant Glioma Neurospheres

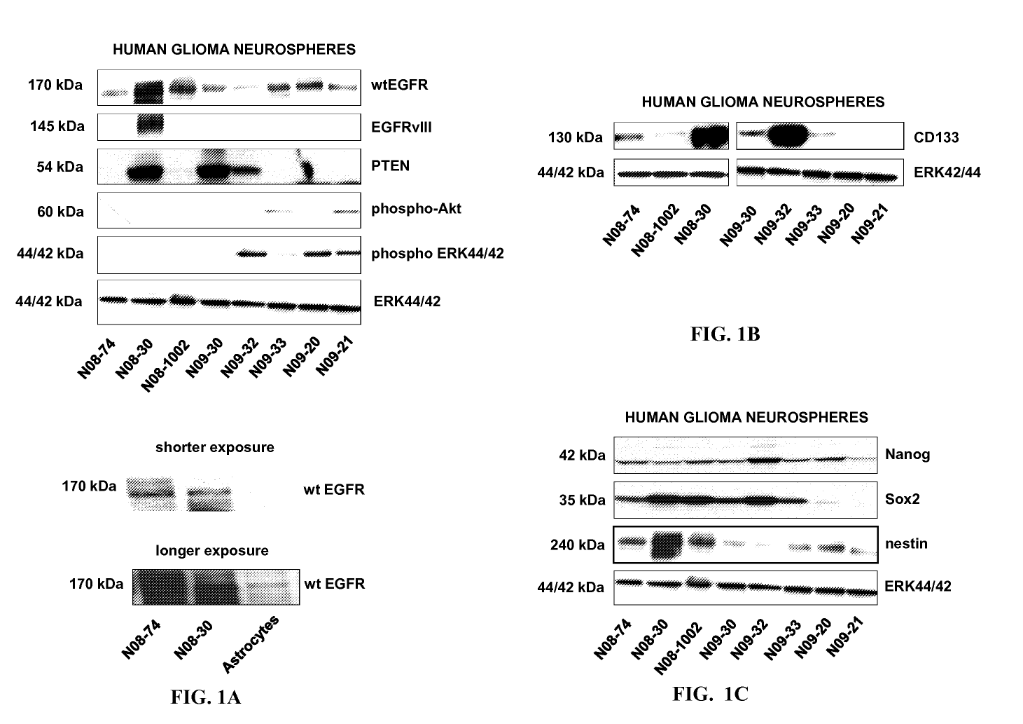

Glioma neurospheres are pathologically relevant models that stably maintain genomic changes of the primary tumor, exhibit stem-like tumor properties, and recapitulate the invasive behavior of gliomas. Neurospheres derived from fresh human surgical specimens of eight GBM patients (passage 5 to 10) were analyzed for the presence of genetic alterations known to occur at high frequency in GBM, such as wtEGFR overexpression or expression of the EGFRvIII mutant. Western blotting confirmed that, relative to normal astrocytes, all neurosphere cultures express higher levels of wtEGFR and that these levels varied in the neurosphere set: N08-30 displayed strong, N08-74, N08-1002, N09-30 N09-33, and N09-21 intermediate, and N08-32 weak expression. Only the N08-30 neurospheres were positive for both wtEGFR and the EGFRvIII mutant (FIG. 1A). To further characterize the neurospheres, activation of the phosphatidylinositol 3-kinase (PI3-K) and extracellular signal-regulated kinase (ERK) pathways and loss of PTEN expression was tested. Phosphorylation of Akt was most prominent in N09-33 and N09-21 neurospheres, whereas phosphorylation of ERK44/42 was the strongest in N09-32, N09-20, and N09-21 neurospheres. The remaining neurospheres showed very low or no phosphorylation of ERK44/42 and Akt (FIG. 1A). PTEN expression was detected in neurospheres N08-30, N09-30, and N09-32 (FIG. 1A). Glioma neurospheres still retain relatively higher expression of wtEGFR (in comparison with normal human astrocytes) after as many as 31 (N08-74) and 46 passages (N08-30) (FIG. 1A, lower panel). For all our experiments, neurospheres in early passage were used. Next, glioma neurospheres were tested for the stem cell marker CD133. Expression of CD133 was the highest in N08-30 and N09-32 neurospheres, whereas neurospheres N09-20 and N09-21 had no detectable CD133 protein (FIG. 1B). All neurospheres were positive for stem cell markers nestin, Nanog, and Sox-2, except for N08-21 which was positive only for nestin and Nanog. N09-33 and N09-20 had low levels of Sox2 (FIG. 1C).

### Multilineage Differentiation and Tumorigenicity of Glioma Neurospheres

Expression of the stem cell marker CD133 in glioma neurospheres was further characterized by flow cytometry. Quantitative FACS analysis showed 3.6% and 9.5% CD133-positive cells in neurospheres N08-74 and N08-30, respectively. N08-74, N08-30, and N08-1002 neurospheres formed invasive tumors in nude athymic mice brains within 4-11 months after implantation as confirmed by MRI and histological examination. All neurospheres tested showed multi-lineage differentiation. After growing in neurobasal medium supplemented with 10% FCS. Neurospheres became positive for glial (GFAP) and neuronal (Tuj 1) differentiation markers. At the same time, no or significantly decreased expression of the neuronal marker Olig 2 and the stem cell marker CD133 was observed.

Preparation, Physicochemical Characterization of Bioconjugated IONPs, and their Cellular Uptake

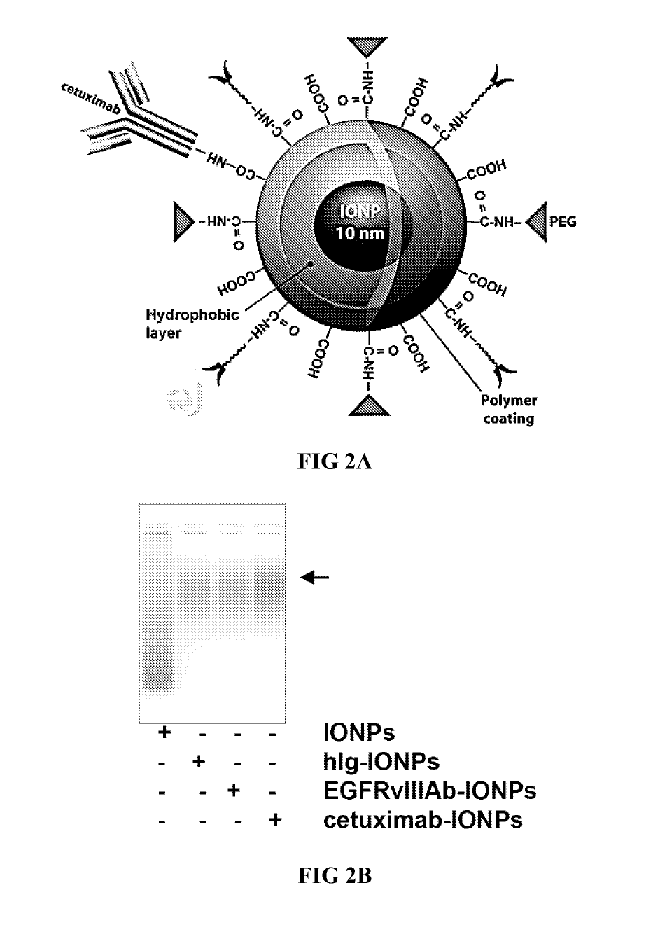

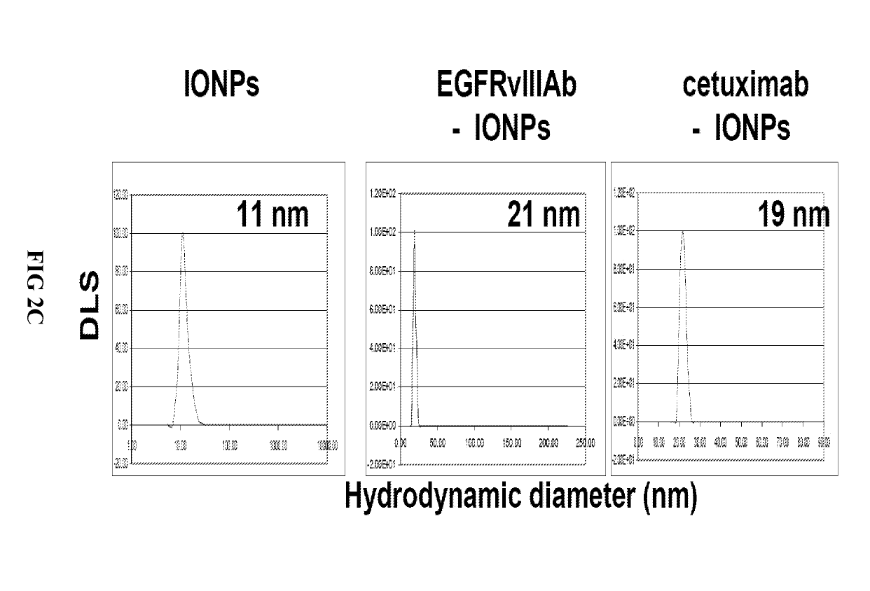

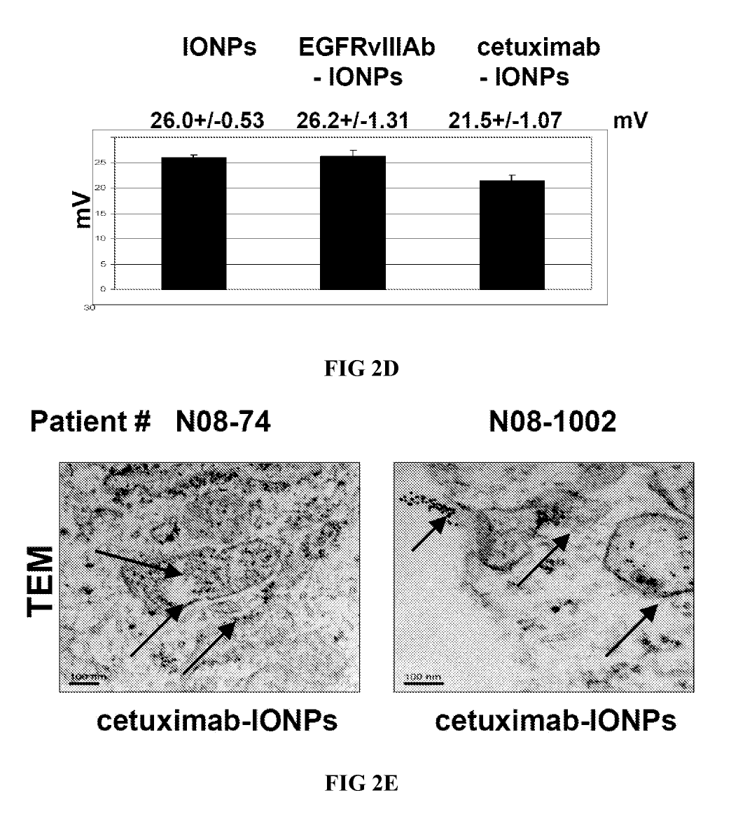

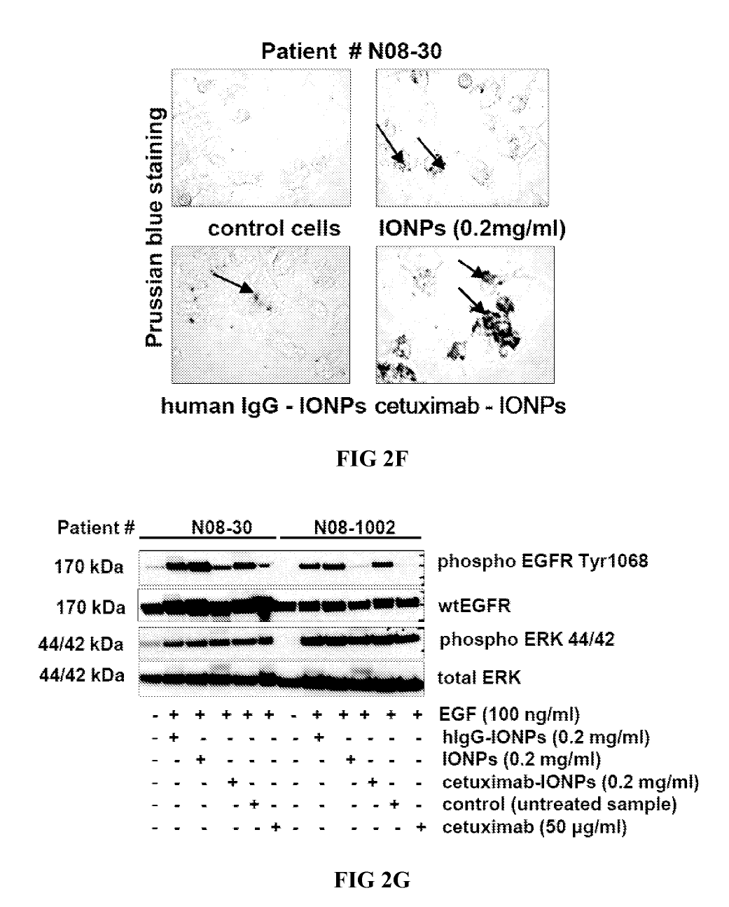

A schematic diagram of the cetuximab-IONPs is shown FIG. 2A. Antibody-conjugated IONPs, prepared by activation of amphiphilic triblock copolymer-coated IONPs (core 10 nm, Ocean NanoTech LLC) in the EDC/NHS solution and covalent conjugation to amino groups of cetuximab, human IgG, and EGFRvIII Abs, had a 1:1 (IONPs: antibody) molar ratio. Conjugation was confirmed by mobility shift on agarose gel electrophoresis (FIG. 2B). Dynamic light scattering (DLS) analysis revealed that the mean diameter of cetuximab-IONPs, EGFRvIIIAb-IONPs, and IONPs nanoparticles was 19, 21, and 11 nm, respectively (FIG. 2C). Zeta potential of cetuximab-IONPs, EGFRvIIIAb-IONPs, and free IONPs was 21.5+/−1.07, 26.2+/−1.31, and 26.0+/−0.53 mV, respectively (FIG. 2D). To evaluate the cellular uptake of cetuximab-IONPs, TEM and Prussian blue staining were performed after treatment of cells. TEM revealed uptake of cetuximab-IONPs by human glioma neurospheres N08-74 and N08-1002. Nanoparticles were found freely within the cytosol and in endosomes, suggesting endocytosis of the cetuximab-IONPs (FIG. 2E). To assess the targeting effect by cell binding, Prussian blue staining of human glioma neurospheres N08-30 incubated were compared with IONPs, hIgG-IONPs, and cetuximab-IONPs for 24 hs. Data in FIG. 2F confirm that the cetuximab-IONPs had the highest uptake, suggesting improved internalization of cetuximab-IONPs, compared to IONPs or hIgG-IONPs. Antibody functionality of the cetuximab-IONPs was tested by evaluating their effect on EGFR signaling in glioma neurospheres. Phosphorylation of Y1068, one of the major autophosphorylation sites in EGFR, is critical for tumorigenicity and indicates EGFR activity. Biological activity of cetuximab-IONPs was documented by markedly decreased phosphorylation of Y1068 in N08-30 and N08-1002 neurospheres treated with cetuximab-IONPs (FIG. 2G).

### Cytotoxic Effect of Cetuximab-IONPs in Human Glioma Neurospheres In Vitro

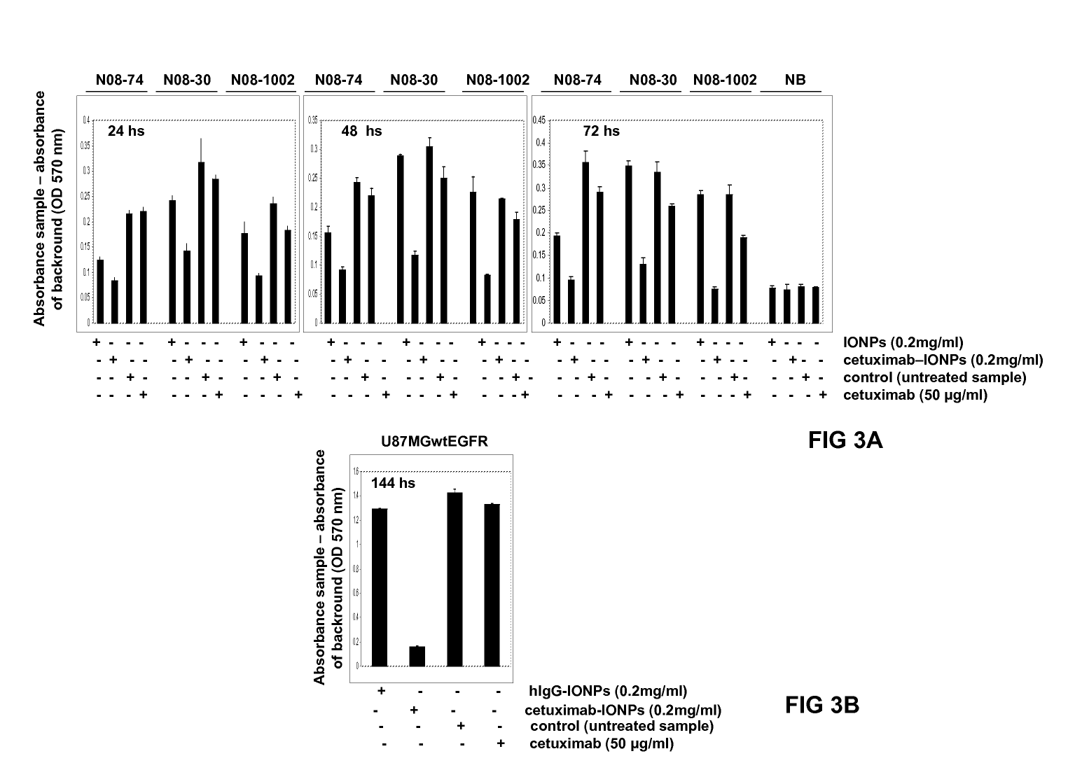

Cetuximab was reported to elicit significant cytotoxicity and increased apoptosis in GBM cell lines with EGFR amplification in a range of concentrations. The effect of the bioconjugate cetuximab-IONPs were tested on the growth of human glioma neurospheres with varying levels of EGFR expression. Neurospheres N08-74, N08-30, and N08-1002 were incubated with free IONPs, cetuximab-IONPs, and cetuximab alone for 24, 48, and 72 hs. Cells from adult normal human brain (NB) were used as a control. A cell proliferation and viability assay was performed after each time point. In this assay, the absorbance of control (untreated neurospheres) cells is considered as 100% of live cells and the absorbance of treated cells is proportionate to that of the control cells. Cetuximab-IONPs significantly decreased survival in all neurospheres tested, most prominently after 72 h treatment: 75% in N08-74, 64% in N08-30, and 80% in N08-1002 (P<0.001). Cetuximab alone had a modest effect on neurosphere cell viability, with a 20% decrease in N08-74, 18% in N08-30, and 28% in N08-1002, whereas none of the treatments were toxic in normal NB cells after 72 hs (FIG. 3A). Cetuximab-IONPs decreased viability of NO8-30 human glioma neurospheres in a dose-dependent manner, in contrast to hIgG-IONPs treatment. These results confirm that the conjugation of cetuximab to IONPs is crucial for the enhanced cytotoxicity found against all glioma neurospheres. Free IONPs did result in a cytotoxic effect with N08-74 neurospheres (about 50% decrease) due to non-specific uptake of free IONPs (data not shown). As an additional control, the malignant glioma cell line U87MGwtEGFR was used. Treatment with hIgG-IONPs, cetuximab-IONPs, control vehicle, or cetuximab for 6 days (144 hs) resulted in a significantly decreased survival (˜90%) only in U87MGwtEGFR cells treated with cetuximab-IONPs (FIG. 3B). Together, these results indicate a significant and selective cytotoxic effect of cetuximab-IONPs on the growth of human glioma neurospheres and cell lines but not with normal brain cells.

### Cetuximab-IONPs Induce Apoptosis and Decrease VEGF Expression in Human Glioma Neurospheres

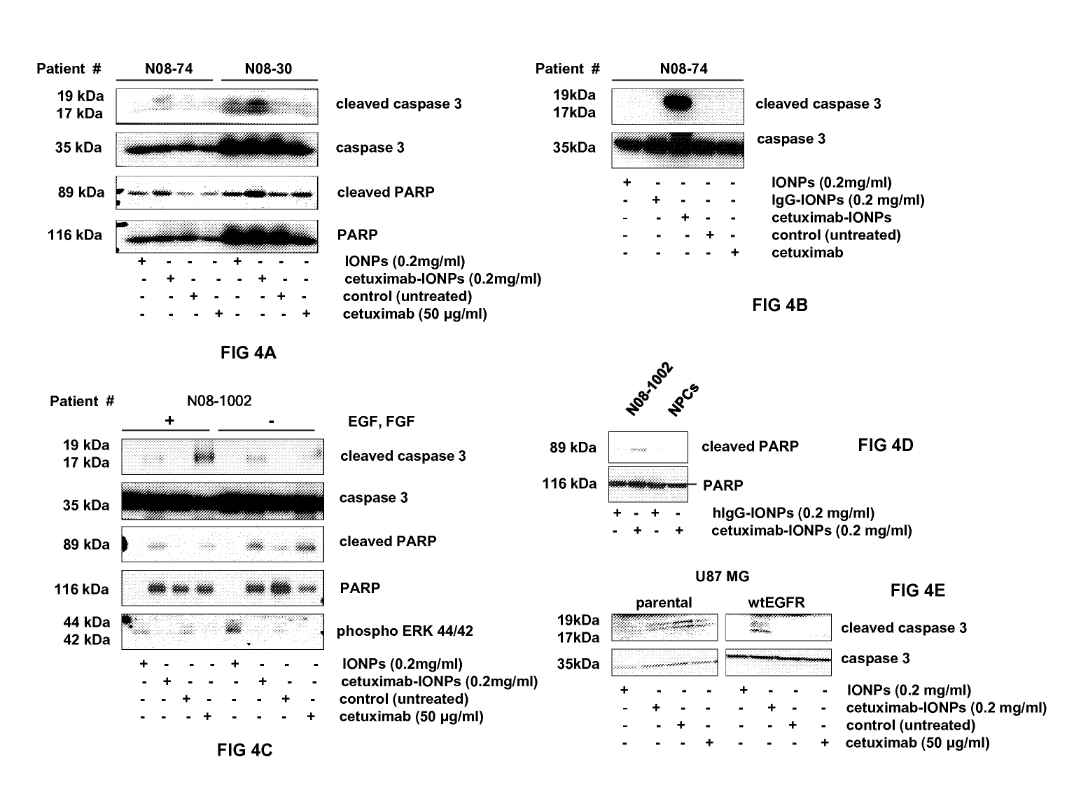

Cytotoxicity of the glioma neurospheres after treatment with the cetuximab-IONPs were examined. Apoptosis as a mechanism of cell death was investigated in addition to autophagy. In human glioma neurospheres N08-74, N08-30, and N08-1002 treated with free IONPs, cetuximab-IONPs, and cetuximab no cleavage of LC3B-I to LCB3-II, the hallmark of authopagy, was observed. This indicated that autophagy is not a likely mode of cell death in glioma neurospheres treated with the cetuximab-IONPs. In contrast, treatment with cetuximab-IONPs resulted in elevated levels of cleaved caspase 3 and cleaved PARP in human glioma neurospheres N08-74, without any cleavage observed in cells treated with free IONPs and cetuximab for 3 hs (FIG. 4A). After a 14 h incubation, induction of caspase 3 cleavage was even more pronounced (FIG. 4B). In neurospheres N08-30, treatment with cetuximab-IONPs also resulted in elevated levels of cleaved PARP and cleaved caspase 3, although free IONPs also increased caspase 3 cleavage (FIG. 4A), most likely due to nonspecific uptake as evidenced in FIG. 2F. On the other hand, in neurospheres N08-1002 both cetuximab-IONPs and cetuximab alone caused apoptosis through cleavage of PARP and caspase 3. Concomitant, EGF and FGF-independent decrease in phospho-ERK44/42 levels were also observed (FIG. 4C), whereas inhibition of Akt phosphorylation was detected only in the absence of growth factors. It is of note that in neurospheres N08-1002, administration of free IONPs completely abrogated expression of PARP, also in a growth factor-independent fashion (FIG. 4C). When comparing human glioma neurospheres N08-1002 and normal human progenitor cells (NPCs), elevated PARP cleavage was observed only in neurospheres treated with cetuximab-IONPs (FIG. 4D). With U87MG cells, cetuximab-IONPs treatment resulted in elevated levels of cleaved caspase 3 only in U87MGwtEGFR cells but not in the parental U87MG cell line deficient in wtEGFR expression (FIG. 4E). These data highlight the importance of EGFR for biological activity of the cetuximab-IONPs.

The effect of cetuximab-IONPs on VEGF production by the glioma neurospheres was tested. Cetuximab-IONPs completely abolished secretion of VEGF in human glioma neurospheres N08-74 and N08-30, whereas cetuximab alone and free IONPs had no effect. In summary, these data demonstrate that cetuximab-IONPs specifically induce increased apoptosis in human glioma neurospheres and cell lines in an EGFR-dependent fashion and significantly decrease VEGF expression.

### Molecular Profile and Characterization of Human GSCs In Vitro and In Vivo

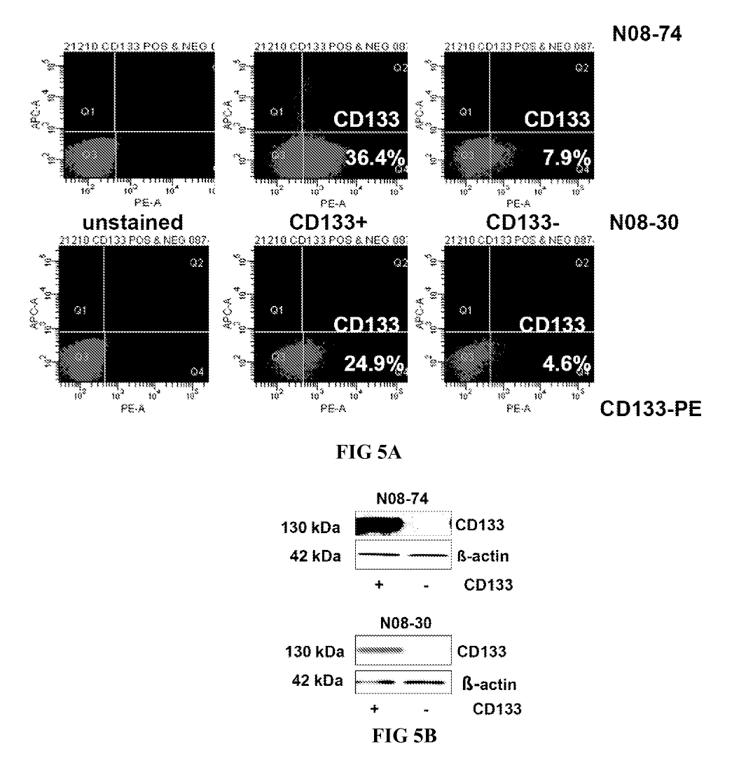

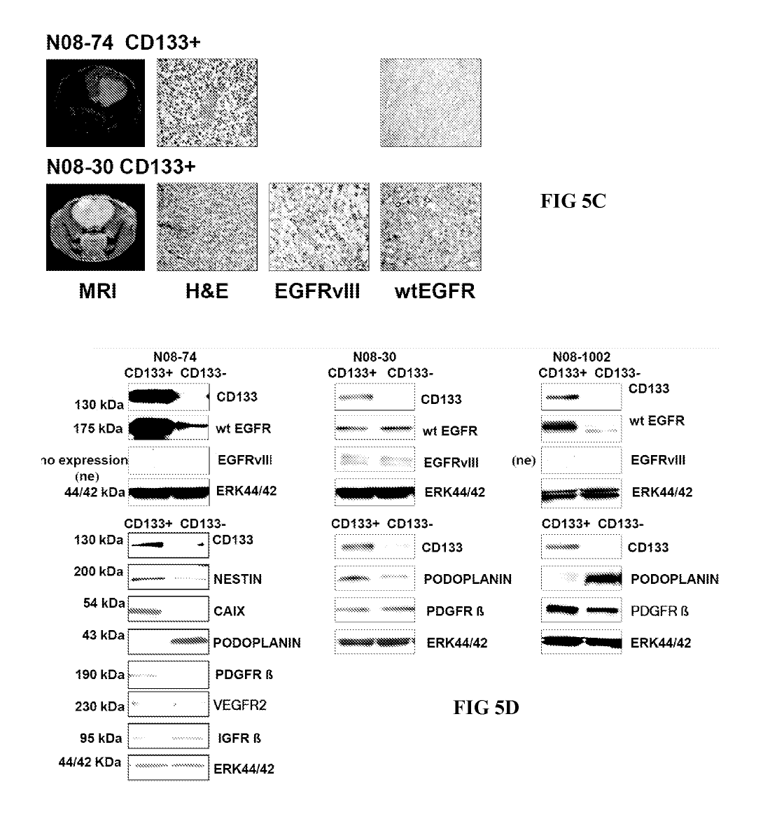

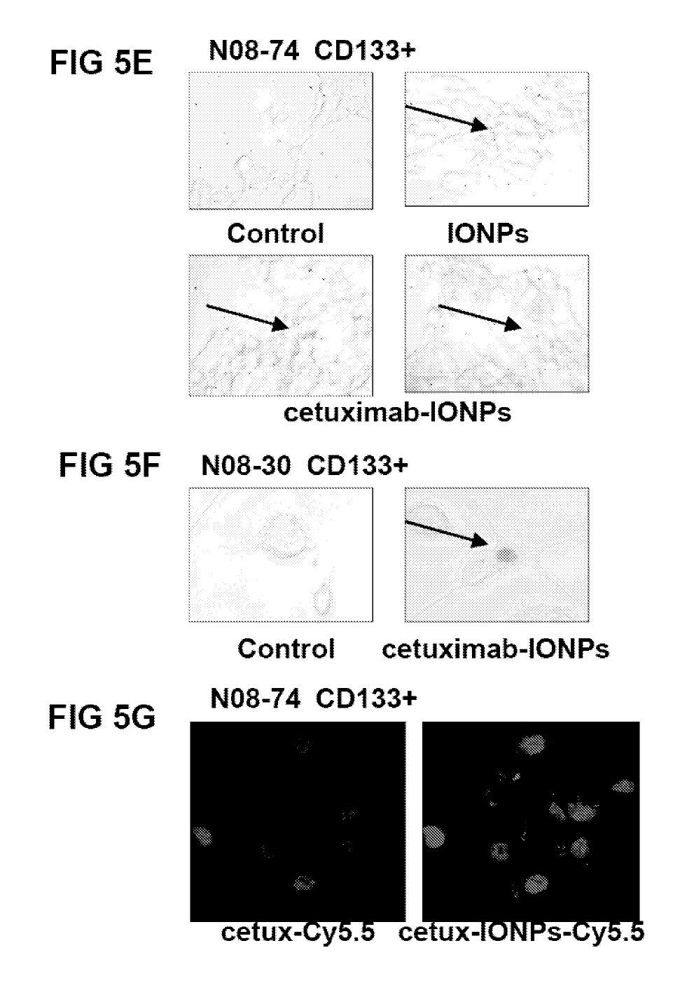

Utilizing FACS analysis, CD133-positive GSCs cells were isolated from human glioma neurospheres N08-74, N08-30 (FIG. 5A), and N08-1002. Following expansion, CD133 expression was verified by FACS analysis and Western blotting (FIG. 5B). CD133-positive GSCs from all tested neurospheres (N08-74, N08-30, and N08-1002) were tumorigenic in athymic nude mice. Mice inoculated with 104 N08-30 GSCs developed invasive intracranial GBM tumors and died within 21 weeks after implantation. Mice inoculated with 105 glioma CD133-negative N08-30 cells (10× more than GSCs) also developed invasive intracranial glioma tumors at a later point. Intracranial glioma tumors were confirmed by MRI and histological examination. Both wtEGFR and EGFRvIII expression in vivo were demonstrated by immunohistochemistry (FIG. 5C). Interestingly, CD133-positive GSCs from neurospheres N08-74 and N08-1002 expressed higher levels of wtEGFR than CD133-negative cells, whereas CD133 status had no effect on wtEGFR and EGFRvIII expression in fractions isolated from neurospheres N08-30 (FIG. 5D).

Overexpression of platelet-derived growth factor (PDGF) A, B and the corresponding TK receptor have been found in all grades of astrocytomas, with the highest incidence (˜25%) in GBM (Fleming et al., 1992; Haberler et al., 2006). In CD133-positive N08-74 GSCs, PDGFRβ, nestin, a tumor biomarker carbonic anhydrase (CA) IX (Kaluz et al., 2009), and podoplanin were overexpressed, as opposed to VEGFR2 and IGFRβ that did not change. Intriguingly, podoplanin was overexpressed in CD133-positive N08-30 GSCs and CD133-negative cells from N08-1002 (FIG. 5D). GSCs thus displayed a molecular signature that is distinguishable from GBM CD133-negative cells.

GSCs retained the multi-lineage differentiation capacity and differentiated into astrocytes and neurons when grown in neurobasal medium supplemented with 10%. This was confirmed by Western blotting that revealed elevated expression of glial (GFAP) and neuronal (Tuj 1) differentiation markers, and the loss of expression of the stem cell marker Olig 2. Unexpectedly, N08-30 GSCs had high basal level of GFAP and Tuj 1 markers. In the medium with serum, human GSCs grew as adherent cultures. Renewal capacity of GSCs was confirmed by plating 1-3 cells in 96 well plate; neurospheres formed after a 14 day incubation.

### GSC Binding and Internalization of Cetuximab-IONPs

Internalization of cetuximab-IONPs in CD133-positive N08-74 and N08-30 GSCs in vitro was confirmed by Prussian blue staining at 24 hs (FIG. 5E,F). Data in FIG. 5E confirm again that the cetuximab-IONPs were most efficiently taken up by the N08-74 GSCs, suggesting improved internalization of cetuximab-IONPs compared to IONPs. In addition, confocal microscopy of N08-74 GSCs treated with Cy5.5-conjugated cetuximab and cetuximab-IONPs for 4 hs was performed. The ratio of pseudo-red (Cy5.5) to pseudo-blue (DAPI) signals confirmed more efficient internalization of cetuximab-IONPs-Cy5.5 compared with cetuximab-Cy5.5 alone (7× higher in the former, FIG. 5G).

### Cytotoxic Effect of Cetuximab-IONPs in Human GSCs In Vitro

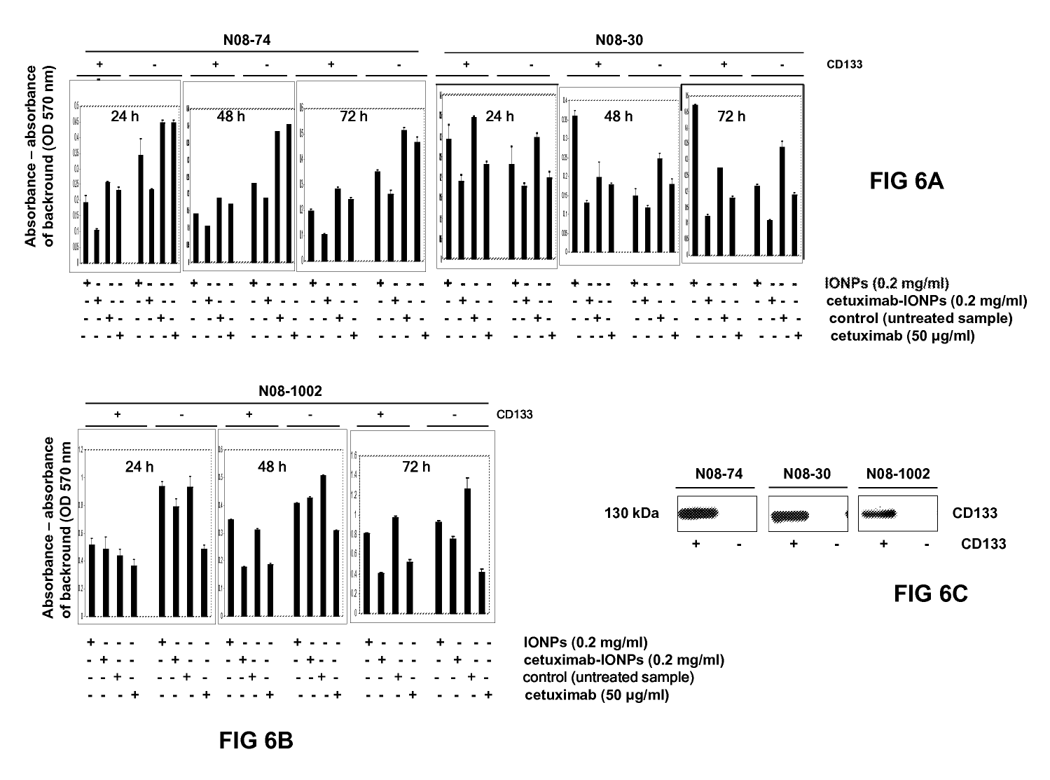

The effect of cetuximab-IONPs on the growth of human GSCs was investigated. GSCs and glioma CD133-negative cells from N08-74, N08-30 (FIG. 6A), and N08-1002 neurospheres with varying levels of EGFR expression (FIG. 6B) were treated with free IONPs, cetuximab-IONPs, and cetuximab for 24, 48, and 72 hs. CD133 positivity in GSCs on the day of the experiment was verified by Western blot (FIG. 6C). A cell proliferation and survival assay revealed that the most profound decrease in survival (after 72 hs) occurred in GSCs treated with cetuximab-IONPs (70% in N08-74, 60% in N08-30, and 60% in N08-1002) and in glioma CD133-negative cells treated with cetuximab-IONPs (50% in N08-74, 60% in N08-30, and 40% in N08-1002) (FIG. 6A,B) (P<0.001). Treatment with cetuximab alone also resulted in decreased cell survival in all GSCs and glioma CD133-negative cells, albeit not as efficient as with the cetuximab-IONPs (FIG. 6A,B). These data confirm a significant cytotoxic effect of cetuximab-IONPs on human GSCs.

### Cetuximab-IONPs Antitumor Effect In Vitro and In Vivo in a Human Malignant Glioma Rodent Model

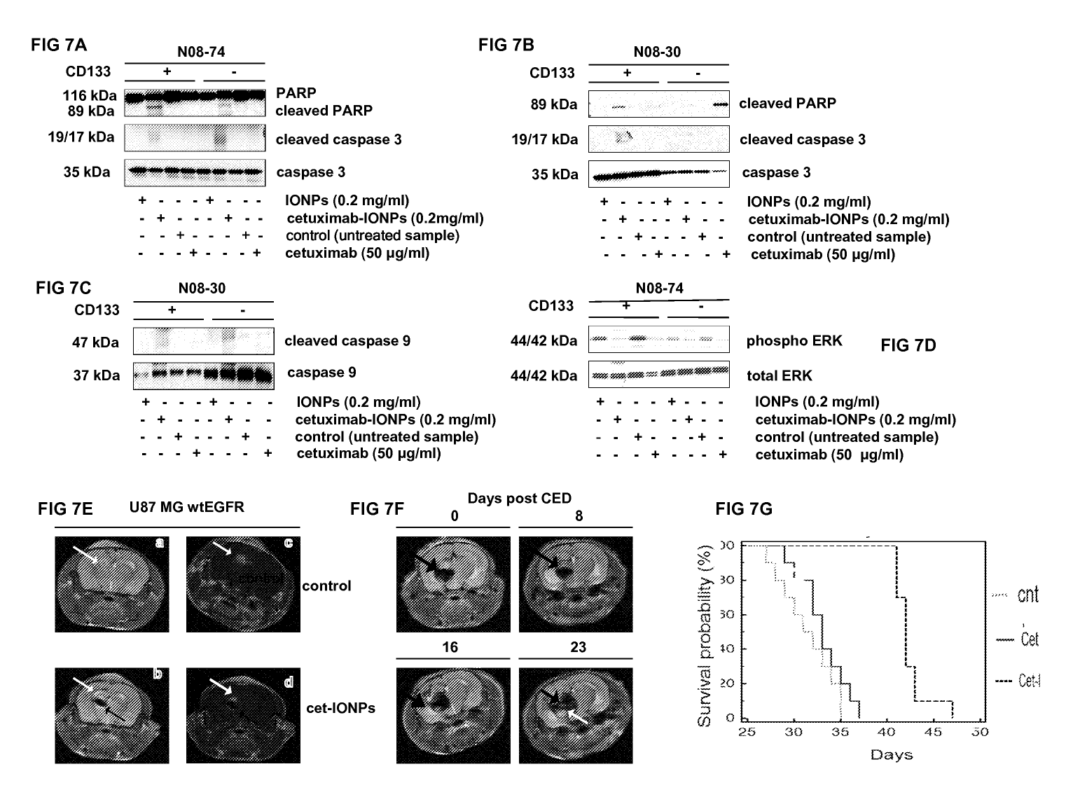

Apoptosis and cleavage of caspase 3 was investigated as a mechanism of cell death in GSCs and glioma CD133-negative cells. GSCs and glioma CD133-negative cells from the neurospheres N08-74 and N08-30 were incubated with free IONPs, cetuximab-IONPs, human IgG, and cetuximab alone. Cetuximab-IONPs induced apoptosis in GSCs and glioma CD133-negative cells, as indicated by the presence of cleaved PARP and caspase 3 after treatment for 3 hs (FIG. 7A) and cleaved PARP after 6, 8, and 24 hs. Increase in cleaved caspase 3 or PARP after treatment with free IONPs and cetuximab alone was not found. Only cetuximab-IONP treated N08-30 GSCs underwent significant apoptosis, as shown by cleaved PARP and caspase 3, whereas cetuximab alone had less of an effect. In CD133-negative glioma cells from N08-30, the opposite was observed: cetuximab alone was more potent that cetuximab-IONPs in inducing cleavage of PARP (FIG. 7B).

Drug-treated cancer cells can undergo apoptosis due activation of intrinsic or extrinsic pathways. Increased cleavage of caspase 9, a hallmark of activation of intrinsic apoptosis, was found in N08-30 GSCs and GBM CD133-negative cells treated with cetuximab-IONPs (FIG. 7C). Furthermore, cetuximab-IONPs and cetuximab alone significantly inhibited phosphorylation of ERK44/42 in N08-74 GSCs, with a more modest effect observed in glioma CD133-negative cells after 3 (FIG. 7D) and 6 hs.

EGFRvIIIAb-IONPs, but not EGFRvIIIAb alone, induced apoptosis through cleavage of caspase 3 in GSCs and GBM CD133-negative cells from N08-74. These results further support the finding that targeting wtEGFR and the EGFRvIII mutant by EGFR-antibody conjugated IONPs triggers cell death via apoptosis in human GSCs.

For in vivo studies, an orthototopic mouse model with the malignant glioma U87MGwtEGFR cell line that expresses the CD133 stem cell marker was used. They are highly tumorigenic in immunocompromised mice. Athymic nude mice implanted with U87MGwtEGFR cells were divided into 4 convection-enhanced delivery (CED) treatment groups, each with 10 animals: cetuximab-IONPs, free IONPs, cetuximab only-treated groups, and a control group. Four additional mice were implanted with malignant glioma cells to verify xenograft tumor growth prior to CED treatment. On day 5, these four mice were sacrificed and the intracranial xenografts were confirmed by histology.

H&E and Prussian blue staining were performed after CED. Immunostaining with wtEGFR antibody confirmed wtEGFR positivity. In addition, the targeting and presence of the nanoparticles in human malignant glioma xenografts was confirmed by MRI (FIG. 7E and F). MRI confirmed T2-weighted signal drop and distribution of cetuximab-IONPs on day 0 after CED. The T2 signal drop increased on days 8, 16, and 23, confirming progressive dispersion of the bioconjugated IONPs into the surrounding brain (FIG. 7F). Dramatic T2-weighted signal drop within the xenograft tumor was observed in mice that underwent cetuximab-IONP CED (FIG. 7E, left lower panel, black arrow). Finally, a significant increase in animal survival was found in mice treated with cetuximab-IONPs. In this group, the median survival time was 42 days, control mice died in 31.5 days. In animals that underwent CED with free IONPs (data not shown) and cetuximab only, the median survival time was 34 days and 33 days respectively (P<0.001) (FIG. 7F). These data demonstrate that cetuximab-IONPs induce apopotosis in GSCs and a significant increase in survival of experimental animals implanted with highly tumorigenic malignant glioma xenografts. This is evidence that cetuximab-IONPs are feasible for in vivo glioma tumor targeting and an antitumor effect.

Canine Spontaneous Glioma Patients Treated with CED of Cetuximab-IONPs

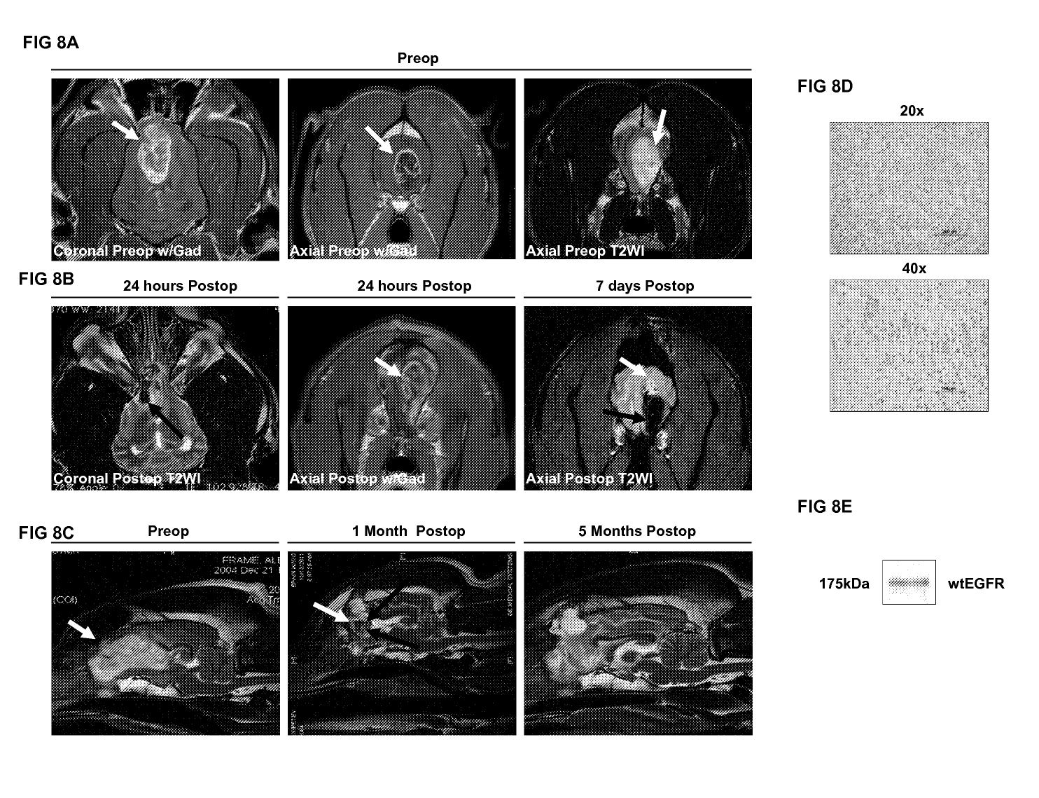

The safety of cetuximab-IONP CED in the healthy canine brain was reported in Platt et al., Clin Neurosurg, 2012, 59:107-113. Gliomas develop spontaneously in the canine, and the majority of spontaneous canine gliomas overexpress EGFR. The therapeutic potential of the cetuximab-IONPs was tested in a spontaneous canine glioma model. The first canine patient presented with a seizure and was found on preoperative MRI to have a large contrast-enhancing tumor after gadolinium administration (white arrow) with pronounced brain mass effect (FIG. 8A,B). The tumor was partially resected after a craniotomy. The canine patient underwent placement of two catheters into the residual tumor on the day of surgery, followed by CED of cetuximab-IONPs. A postoperative MRI at 24 hs, 7 days, and 1 month showed residual tumor (white arrow) and T2 signal drop correlating with intra- and peri-tumoral cetuximab-IONPs (black arrows) (FIG. 8A,B,C). Postoperative imaging of the canine glioma patient revealed a marked antitumor response 5 months after cetuximab-IONP CED treatment (FIG. 8C, last MRI). The histopathological analysis classified the canine tumor as a WHO grade 2 oligodendroglioma with anaplastic features consisting of mitotic activity (FIG. 8D). Western blot analysis of the tumor did reveal expression of wtEGFR (FIG. 8E). The canine patient showed no signs of toxicity, nor evidence of neurological impairment after CED treatment and showed seizure-free treatment for 15 months ago. Another canine with a spontaneous glioma (WHO grade II oligodendroglioma) was also treated in a similar fashion and also remained alive and well without seizures for 14 months after CED treatment. Our data in a spontaneous canine glioma model provide further evidence of the targeting and antitumor effect of cetuximab-IONPs and suggest their potential therapeutic application in humans.

### Generation of Human Malignant Glioma Neurospheres

Tumor specimens were collected from patients with a histologic diagnosis of GBM (WHO Grade IV astrocytoma). Confirmation of tumor diagnosis and grading was performed by neuro-pathologists at Emory University. Patient tumor specimens (patients # N08-74, N08-1002, N08-30, N09-30, N09-32, N09-33, N09-20, N09-21) were harvested at the time of surgical resection with approval by the Emory University Institutional Review Board (IRB) (protocol 642-2005). Samples were minced with a scalpel and tissue fragments were digested for 30 min at 37° C. with 1 mg/ml collagenase/Dispase (Roche) and separated by Ficoll gradient. Malignant glioma neurospheres were cultured in a Neurobasal A-medium with N-2 and B-27 supplements (Invitrogen), 10 ng/ml human recombinant bFGF and 20 ng/ml EGF (both STEMCELL Technologies) at 37° C. and 5% CO₂. In all experiments, neurospheres were used in early passage.

### Isolation of Human Glioma CD133-Positive Cells Using FACS

Neurospheres (passage 5 to 10) were dissociated using Accutase (Chemicon) and 1-8×10⁷ cells in single cell suspensions were stained with anti CD133/1 (AC133)-phycoerythrin (PE)-coupled antibody (Miltenyi Biotech) and sorted using FACS Vantage SE (Becton Dickinson). In all experiments, human glioma CD133-positive and negative cells were used in early passage.

### Cell Lines, Human Orthotopic Xenografts, and Antibodies

The human malignant glioma cell lines U87MG (ATCC, HTB-14 TM) and U87MGwtEGFR (stably overexpressing wtEGFR) are known. Expression of wtEGFR was verified by Western blot. Human astrocytes (ScienCell Research Laboratories) and normal human progenitor cells (Lonza) were grown as recommended by suppliers. Normal human adult brain tissue, obtained from patients undergoing epilepsy surgery at Emory University (IRB protocol 642-2005), was cultured in DMEM/F12 (50/50 mix, Cellgro) and 10% FCS (HyClone) in the presence of sodium pyruvate, Penstrep, and L-glutamine (HyClone). Each cell line was used in early passage. All human orthotopic malignant glioma xenograft studies were performed in six to eight week old nude female athymic (nu/nu) mice after approval by the Institutional Animal Care and Use Committee of Emory University. Antibodies used for bioconjugation were: cetuximab (erbitux; Imclone LLC; kindly provided by the Winship Cancer Institute of Emory University pharmacy), rabbit anti EGFRvIIIAb (GenScript Corp.), and human IgG (Bethyl Laboratories, Inc., Texas, USA).

### Antibody Bioconjugation of IONPs

IONPs were purchased from Ocean NanoTech (Arkansas, USA). For conjugation, carboxyl groups on the IONPs were activated in an activation buffer, provided by the manufacturer (ethyl dimethylaminoprolyl carbodiimide (EDC) and sulfo-N-hydroxysuccinimide (NHS). The EDC/NHS solution was mixed vigorously with IONPs at 25° C. for 20 min. The IONPs with activated carboxyl groups (100 μl at 5 mg/ml) were then reacted with antibodies (62.5 μl at 2 mg/ml) at 25° C. for 2 hs and the reaction mixtures were stored at 4° C. overnight. Excess antibody was removed by three rounds of centrifugation using 300K MWCO OMEGA membranes, followed by resuspending IONPs in PBS. Conjugation was visualized using mobility shift in 1% agarose gel.

### Physicochemical Characterization of Bioconjugates

Hydrodynamic size and Zeta potential of the bioconjugated IONPs were measured by the ZetaSizer Nano. Bioconjugates were prepared in a washing buffer (Ocean NanoTech) at 1 mg/ml and sonicated for 5 min. Measurements were performed at 25° C.

Conjugation of Cetuximab-IONPs with Cy5.5 and Confocal Microscopy

Cy5.5 was conjugated to cetuximab and cetuximab-IONPs with EasyLink APC/Cy5.5 conjugation kit (Abcam, Mass., USA). Single cell suspensions of GSCs N08-74 (2×104/8 well chamber slide) were treated with cetuximab-IONPs-Cy5.5 (0.2 mg/ml) and cetuximab-Cy5.5 (50 μg/ml) for 4 hs, fixed, and imaged using Zeiss LSM 510 Meta Confocal microscope. Quantification was performed using Zen 2011 Light Edition.

### Tumor Inoculation and CED

Anesthetized athymic nude mice were placed in a stereotactic instrument and U87wtEGFR malignant glioma cells (5×10⁵ resuspended in DMEM) were stereotactically inoculated into the right striatum, 3 mm below the cortical surface (day 0). On day 5 after tumor implantation, mice were randomized into four groups (10 in each group) for CED of: a) untreated control; b) free IONPs; c) cetuximab only; and d) cetuximab-IONPs. The CED infusion apparatus consisted of a hydraulic drive serially connected to a digital syringe pump controller (UltraMicroPumpII, World Precision Instruments, Inc., Sarasota, Fla.). CED was performed with 5 μg total dosage in a 10 μl volume (0.5 mg/ml) at a rate of 0.5 μl/min (20 min of total infusion).

### Imaging

Anesthetized mice underwent MRI scanning on a 4.7-T animal MRI scanner using a dedicated mouse coil (Varian Unity). T2 weighted fast spin echo sequences with TR/TE=6500/70 ms were typically used for imaging of the tumor and IONPs in the brain. All MRI scans for the canine patient were obtained in the Bioimaging Research Center (BIRC) at the University of Georgia, College of Veterinary Medicine (CVM-UGA) on a 3 T MRI unit (GE Signa HDx). Under general anesthesia, dogs were placed in sternal recumbency. The head was immobilized in the extremity coil and positioned in the isocenter of the magnet. Whole brain scans were performed with T1-weighted fast spin echo sequence, T2-weighted fast spin echo sequence, T1 and T2 weighted fluid attenuated inversion recovery (FLAIR) sequence and 3D gradient echo sequence, before and after IONP CED. Since the size of the canine brain approaches that of the human brain, MRI parameters selected for each canine were slightly modified from those of the brain MRI protocol used in human patients at Emory University. The field of view (FOV) of 16 cm, matrix of 256 by 256, slice thickness of 3.5 mm (0.5 mm gap) were used in the scan sequences. The scans were performed in the transverse planes.

### Spontaneous Canine Glioma CED Treatment

Treatment of the first canine patient with a spontaneous glioma was completed at the University of Georgia, College Veterinary Medicine (CVM-UGA) by the Neurosurgery service with an approved CVM-UGA IACUC protocol (A2010 4-055). All histopathological slides were evaluated by an Emory neuropathologist C.H. The canine patient underwent a craniectomy and partial resection of an EGFR-expressing WHO grade II oligodendroglioma with anaplastic features. At surgery, two intrathecal catheters (Medtronic Inc., MN, USA) were implanted within the residual tumor and tunneled subcutaneously through the skin. Each catheter was connected to an external programmable Medtronic Synchromed II pump secured on the dorsal surface of the canine head with bandage wrapping. The patient underwent a 3 day CED infusion (0.5 μl/min) of cetuximab-IONPs (0.5 mg/ml; total volume of 2.16 ml). The canine patient was able to move freely within his living space and did not require any anesthesia during the CED procedure. MRI was used to confirm intratumoral positioning of CED catheters and initial distribution of cetuximab-IONPs. Follow-up MRI scans were obtained to assess therapeutic efficacy. Routine clinical follow-up was also performed with the patient and complete physical examinations and routine blood work were performed. A second canine patient with a spontaneous glioma underwent CED treatment in the same manner.

What we claim:

1. A method of treating brain cancer comprising administering an effective amount of a pharmaceutical composition to a human subject in need thereof wherein the pharmaceutical compositions comprises a nanoparticle conjugated to a molecule that binds to the extra-cellular domain of the human EGF receptor.

2. The method of claim 1, wherein the molecule is an antibody, antibody fragment, or antibody mimetic.

3. The method of claim 1, wherein the molecule is cetuximab.

4. The method of claim 1, wherein the molecule is human/mouse chimeric monoclonal antibody wherein the antibody comprises human IgG1 heavy and kappa light chain constant regions produce by a mammalian cell culture.

5. The method of claim 4, wherein the antibody comprises the following CDR1, CDR2, and CDR3 sequences in a variable heavy region (SEQ ID NO: 1) Ser Tyr Trp Ile Glu, (SEQ ID NO: 2) Glu Ile Leu Pro Gly Ser Lys Lys Thr Asn Tyr Asn Glu Lys Phe Lys Gly, and (SEQ ID NO: 3) Tyr Tyr Tyr Arg Asn Asp Asp Tyr Gly Met Asp Tyr.

6. The method of claim 4, wherein the antibody comprises the following CDR1, CDR2, and CDR3 sequences in a variable light region (SEQ ID NO: 4) Ser Ala Ser Gln Asp Ile Arg Asn Tyr Leu Asn, (SEQ ID NO: 5) Tyr Thr Ser Thr Leu His Ser, and (SEQ ID NO: 6) Gln Gln Tyr Ser Lys Ile Pro Tyr Thr.

7. The method of claim 1, wherein the antibody comprises the following sequence in a variable heavy region (SEQ ID NO: 7) Gln Val Gln Leu Gln Gln Ser Gly Ala Glu Leu Met Lys Pro Gly Ala Ser Val Lys Ile Ser Cys Lys Ala Thr Gly Tyr Thr Phe Ser Ser Tyr Trp Ile Glu Trp Val Lys Gln Arg Pro Gly His Gly Leu Glu Trp Ile Gly Glu Ile Leu Pro Gly Ser Lys Lys Thr Asn Tyr Asn Glu Lys Phe Lys Gly Lys Ala Thr Phe Thr Ala Asp Thr Ser Ser Asn Thr Ala Tyr Met Gln Phe Ser Ser Leu Thr Ser Glu Asp Ser Ala Val Tyr Tyr Cys Ala Arg Tyr Tyr Tyr Arg Asn Asp Asp Tyr Gly Met Asp Tyr Trp Gly Gln Gly Thr Ser Val Thr Val Ser Ser.

8. The method of claim 4, wherein the antibody comprises the following polypeptide sequence in the variable light region (SEQ ID NO: 8) Glu Ile His Met Thr Gln Thr Thr Ser Ser Leu Ser Ala Ser Leu Gly Asp Arg Val Thr Ile Ser Cys Ser Ala Ser Gln Asp Ile Arg Asn Tyr Leu Asn Trp Tyr Gln Gln Lys Pro Asp Gly Thr Val Lys Leu Leu Ile Tyr Tyr Thr Ser Thr Leu His Ser Gly Val Pro Ser Arg Phe Ser Gly Ser Gly Ser Gly Thr Asp Tyr Ser Leu Thr Ile Ser Asn Leu Glu Pro Glu Asp Ile Ala Thr Tyr Tyr Cys Gln Gln Tyr Ser Lys Ile Pro Tyr Thr Phe Thr Gly Gly Thr Lys Leu Glu Ile Lys Arg Ala Asp Ala Ala.

9. The method of claim 1, wherein the pharmaceutical composition is administered intracranially.

10. The method of claim 9, wherein the pharmaceutical composition is administered by a catheter with an outer diameter of less than 1 mm.

11. The method of claim 1, wherein the particles are administered at greater than or about 0.25 μg/min.

12. The method of claim 1, wherein the subject is diagnosed with a brain tumor.

13. The method of claim 12, wherein the tumor is a glioma or glioblastoma.

14. The method of claim 1, further comprising the step of exposing the brain of the subject to an alternating magnetic field under conditions such that heat is generated about the area of the particles.

15. The method of claim 1, wherein the particles are iron oxide particles or iron particles with a substantially unoxidized core.

16. The method of claim 1, wherein the pharmaceutical composition further comprises a second anticancer agent.

17. The method of claim 16, wherein the second anti-cancer agent is temozolamide, bevacizumab, procarbazine, lomustine, vincristine, gefitinib, erlotinib, docetaxel, cis-platin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, taxol, taxotere, etoposide, teniposide, amsacrine, topotecan, camptothecin, bortezomib, anagrelide, tamoxifen, toremifene, raloxifene, droloxifene, iodoxyfene, fulvestrant, bicalutamide, flutamide, nilutamide, cyproterone, goserelin, leuprorelin, buserelin, megestrol, anastrozole, letrozole, vorazole, exemestane, finasteride, marimastat, trastuzumab, cetuximab, dasatinib, imatinib, combretastatin, thalidomide, and/or lenalidomide or combinations thereof.

18. A pharmaceutical composition comprising a nanoparticle conjugated to a molecule that binds to the extra-cellular domain of the human EGF receptor.

19. The pharmaceutical composition of claim 18 in the form of an aqueous buffer, pill, capsule, or tablet.

20. The pharmaceutical composition of claim 18, wherein the molecule that binds to the extra-cellular domain of the human EGF receptor is cetuximab.
